(19) United States
(12) Patent Application Publication
(10) Pub. No.: US 2019/0326002 A1
(43) Pub. Date: Oct. 24, 2019

(54) SYSTEMS AND METHODS FOR MODIFYING ADAPTIVE DOSING REGIMENS

(71) Applicant: Diane R. Mould, Fort Myers, FL (US)

(72) Inventor: Diane R. Mould, Fort Myers, FL (US)

(21) Appl. No.: 16/391,950

(22) Filed: Apr. 23, 2019

Related U.S. Application Data
(60) Provisional application No. 62/661,305, filed on Apr. 23, 2018.
(60) Provisional application No. 62/815,825, filed on Mar. 8, 2019.

(51) Int. Cl. G16H 20/10; G16H 10/60; A61B 5/00

(52) U.S. Cl. CPC G16H 20/10; G16H 10/60; G16H 10/40; A61B 5/4836; A61B 5/0002

(57) ABSTRACT

The systems and methods described herein determine a patient-specific pharmaceutical dosing regimen for a patient, using a computerized pharmaceutical dosing regimen recommendation system, and provide ways to quickly adjust a dosing regimen recommendation for a specific patient, in order to react to fast changes in the patient's medical condition. One way that the present disclosure accomplishes this is to update various inputs to the model in particular ways, such as by excluding a specific type (or types) of data or a specific portion (or portions) of previously observed data. Excluding this data from the model inputs allows for other data (such as data that is consistent with the indicators of the patient's status) to be weighted more heavily by the model, and therefore results in a recommendation for a patient-specific pharmaceutical dosing regimen that is suitable for quickly addressing the patient's changing needs.

### REFERENCE TO RELATED APPLICATIONS

This application claims priority to and the benefit of U.S. Provisional Patent Application No. 62/661,305, filed on Apr. 23, 2018, and entitled “SYSTEMS AND METHODS FOR MODIFYING ADAPTIVE DOSING REGIMENS” and U.S. Provisional Patent Application No. 62/815,825, filed on Mar. 8, 2019, and entitled “SYSTEMS AND METHODS FOR DRUG-AGNOSTIC PATIENT-SPECIFIC DOSING REGIMENS”. The entire contents of the above-referenced applications are incorporated herein by reference.

### TECHNICAL FIELD

This disclosure relates generally to patient-specific dosing and treatment recommendations including, without limitation, computerized systems and methods that use mathematical models specific to a drug or a class of drugs and observed patient-specific responses to treatment, to predict, propose, modify and evaluate suitable medication treatment plans for a specific patient.

### BACKGROUND

A physician's decision to start a patient on a medication-based treatment regimen involves determination of a dosing regimen for the medication to be prescribed. Different dosing regimens are appropriate for different patients having differing patient factors. By way of example, dosing quantities, dosing intervals, treatment duration and other variables may vary across different dosing regimens. For example, a patient with low neutrophil counts may require a delayed dose, a lower dose than a typical patient, or both. Traditionally, a physician prescribes an initial dosing regimen based on the package insert (PI) for a drug and the physician's own personal clinical experience. After an initial period of treatment, a physician may follow-up with the patient to reevaluate the patient and reconsider the initial dosing regimen. The PI sometimes provides quantitative indications for increasing or decreasing a dose, or increasing or decreasing a dosing interval. Based on both the physician's assessment of the patient, his clinical experience, and the PI information, the physician would adjust the dosing regimen on an ad hoc basis. Adjusting the dosing regimen for a patient is largely a trial and error process, informed by physicians' experience and clinical judgment.

A proper dosing regimen may be highly beneficial and therapeutic, while an improper dosing regimen may be ineffective or even deleterious to the patient's health. Further, both under-dosing and overdosing generally results in a loss of time, money and/or other resources, and increases the risk of undesirable outcomes. Computerized dosing recommendation systems may assist medical professionals in providing and assessing dosing regimens. Medical conditions of patients can change quickly, particularly in the severely ill, and there is a need for dosing regimens to be adjusted quickly to account for these changes.

### SUMMARY

The systems and methods described herein determine a patient-specific pharmaceutical dosing regimen for a patient, using a computerized pharmaceutical dosing regimen recommendation system. A computational model, such as a Bayesian model may be used to determine dosing regimen recommendations. For example, each iteration of the model may include a calculation or a determination of a recommended dosing regimen. When additional data is made available (such as physiological parameter data or drug concentration data obtained from the patient), another iteration of the model may be performed to determine an updated recommended dosing regimen based on the additional data. This process may be repeated any number of times to reflect any new data that describes the patient.

As used herein, a “dosing regimen” includes at least one dose amount of a drug or class of drugs and a recommended schedule for administering the at least one dose amount of the drug to a patient. The dose amount may be a multiple of an available dosage unit for the drug. For example, the available dosage unit could be one pill or a suitable fraction of a pill that results when it is easily split, such as half a pill. In some implementations, the dose amount may be an integer multiple of the available dosage unit for the drug. For example, the available dosage unit could be a 10 mg injection or a capsule that cannot be split. For some routes of administration (e.g., IV and subcutaneous), any portion of the dose strength can be administered. The recommended schedule includes a recommended time for administering a next dose of the drug to the patient, such that a predicted concentration time profile of the drug in the patient in response to the first pharmaceutical dosing regimen is at or above the target drug exposure level (e.g., a target drug concentration trough level) at the recommended time.

In some implementations, the systems described herein may not be specific to a particular drug but instead apply to a class, or subset or grouping of drugs used in a drug-agnostic model. As used herein, the term “drug” may refer to a single drug or a class or set of drugs. A class of drugs is a group of drugs larger than one, which exhibits at least one similar pharmacokinetic (PK) and/or pharmacodynamic (PD) behavior, share a common mechanism of action, or a combination thereof. As an example, a set of drugs may treat the same disease or be used for the same indication, examples of which include general inflammatory disease, inflammatory bowel disease (IBD), ulcerative colitis, Crohn's disease, rheumatoid arthritis, ankylosing spondylitis, psoriatic arthritis, psoriasis, asthma, or multiple sclerosis. A set of drugs may have a similar chemical structure. For example, a set of drugs could include monoclonal antibodies (mAbs), anti-inflammatory compounds, corticosteroids, immunomodulators, antibiotics or biologic therapies. A user such as a doctor, clinician, or a user building a drug-agnostic model may define a class of drugs based on specific criteria, and members of that class may be electronically designated in a database as being part of that class. That database is accessible to systems and methods disclosed herein, for use in determining a class-based dosing regimen that could be used for any drug in the class. In some implementations, a dosing regimen output from the model may not be specific to a single drug but may be generic to the class of drugs, and suitable for any drug in that class. For example, a dosing regimen may include a drug-agnostic unit measurement (e.g., one unit, two units, three units, etc, where a unit corresponds to a specified amount of an active agent) and a time or times for administration.

The development and application of drug-agnostic models allow greater utility for a single model. Rather than implementing multiple models, where each model corresponds to a single drug in a class, a drug-agnostic model is applicable to all drugs within the class and may accordingly be applied to a wider range of patients than single-drug models and across a range of administration routes. For example, the models described herein may include a pharmacokinetic drug-agnostic model capable of being used for all biologics used in the treatment of inflammatory diseases. Such a model can be used to propose dose regimens for fully human monoclonal antibodies (mAb), chimeric mAbs, fusion proteins, and mAb fragments (i.e., a range of drugs with differing pharmacokinetic properties but other similarities such as similar molecular weight and indication) using the same model. The model can be used in a broad patient population, including inflammatory bowel disease, rheumatoid arthritis, psoriatic arthritis, psoriasis, multiple sclerosis, and other such diseases that arise from immune dysregulation. The development and application of drug-agnostic Bayesian models for agents in other broad drug sets (e.g., the aminoglycoside antibiotics, chemotherapeutic agents that cause low white cell counts, etc.) is similarly feasible. Drugs within the class may be administered through a variety of routes, such as subcutaneous, intravenous, oral, intramuscular, intrathecal, sublingual, buccal, rectal, vaginal, ocular, nasal, inhalation, nebulization, cutaneous, or transdermal. Drug-agnostic models may account for route of administration by taking the route of administration as a variable input to the system, allowing greater flexibility for the model.

Because the drug-agnostic model applies to a set of drugs, rather than only a single drug, the model may retain patient-specific information when a patient is treated with multiple drugs within the set of drugs. For example, the set of drugs may include infliximab, vedolizumab, adalimumab, and other anti-inflammatory biologics. If a patient is treated with one drug (e.g. infliximab), then later treated with another drug (e.g. vedolizumab), the model may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the drug-agnostic model to accurately anticipate the patient's ability to process a drug and thereby provide more suitable, patient-specific dosing regimens when a patient changes drug therapy.

The systems and methods described herein provide ways to quickly adjust a dosing regimen recommendation for a specific patient, as a way to react to rapid changes in the patient's medical condition. One way that the present disclosure accomplishes this is to update various inputs to the model in particular ways, such as by excluding a specific type (or types) of data or a specific portion (or portions) of previously observed data. To determine exactly which type or which portion of data to exclude from the model inputs, the systems and methods described herein may rely on indicators of the patient's status, which may be determined and provided by a physician to the system, or automatically assessed by the system itself. As an example, the patient's status may be determined from physiological parameters assessed for the patient, which are discussed in detail below. Depending on the patient's status (such as whether the patient's physiological parameters indicate that the patient is reacting in a desirable manner to drug treatment, for example), certain type(s) or portion(s) of data may be excluded from the model inputs, such as data that is inconsistent with indicators of the patient's current status. Excluding this data from the model inputs allows for other data (such as data that is consistent with the indicators of the patient's status) to be weighted more heavily by the model, and therefore results in a recommendation for a patient-specific pharmaceutical dosing regimen that is suitable for quickly addressing the patient's changing needs.

Generally, the type of data that may be excluded from the model inputs includes drug concentration data, which may reflect one or more physical measurements of an amount of drug in samples obtained from the patient, such as blood. The drug concentration data represents an amount of drug remaining in the patient's body, and is expected to decline at a rate depending on a patient's specific clearance or elimination rate. As used herein, an “elimination rate” refers generally to a rate at which a patient's or animal's body eliminates or clears a drug. Different patients have different elimination rates, which are dependent on the patients' specific physiology and pharmacokinetics. Patients with high elimination rates have drug concentration time profiles (e.g., the drug concentration in the patient's body as a function of time) that decline rapidly, meaning that the effectiveness of the drug is lost relatively quickly over time. In contrast, patients with lower elimination rates have drug concentration time profiles that decline slowly, meaning that the drug remains in the patient's body for longer periods of time. For many drugs, it is generally desirable to maintain an exposure to the drug that is nearly constant, within a specific range, or above a minimum exposure such as a trough value. Because the elimination rate is so dependent on the patient's specific physiology and pharmacokinetics, elimination rates can vary greatly across different patients, and physicians often face difficulty in determining an appropriate dosing regimen for maintaining an adequate exposure to the drug in individual patients.

Bayesian computational models generally take into account all available information, including every available drug concentration measurement data for a patient in drug therapy. The systems and methods described herein differ from typical Bayesian systems in that some data may be expressly excluded from the inputs to the model, which performs dosing regimen calculations. Excluding a certain type or portion of data enables the systems and methods of the present disclosure to react quickly to a patient's change in health, particularly in the severely ill, to adequately address the patient's needs. As an example, if a patient takes a sudden and unexpected turn for the worse and/or the patient's disease is progressing faster than expected, then older concentration data or the entire set of concentration data may be excluded from the model inputs entirely. In this way, the systems and methods described herein place more weight and importance on more recent or more reliable measurements, thereby allowing the system to react quickly to a patient sudden decline in health.

Inputs into the system may be used to update and refine the model for a specific patient taking a specific drug. Inputs into the systems described herein may include concentration data, physiological data, and a target response. The inputs to the model generally include concentration data, physiological data, and a target response. As discussed above, the concentration data is indicative of one or more concentration levels of a drug in one or more samples obtained from the patient, such as blood, blood plasma, urine, hair, saliva, or any other suitable patient sample. The concentration data may reflect a measurement of the concentration level of the drug itself in the patient sample, or of another analyte in the patient sample that is indicative of the amount of drug in the patient's body. The drug may be part of a treatment plan to treat a patient with a particular health condition, such as a disease or disorder like inflammatory bowel disease (IBD, including ulcerative colitis and Crohn's disease), rheumatoid arthritis, psoriatic arthritis, ankylosing spondylitis, plaque psoriasis, or any other suitable affliction. Drugs used to treat such health conditions may include monoclonal antibodies (mAbs), such as infliximab or adalimumab. While many of the examples described herein are with reference to using infliximab to treat IBD, it will be understood that the systems and methods of the present disclosure are applicable to any drug or treatment that loses its effectiveness over time in a measurable way, and may be used to treat any number of diseases, including any inflammatory disease, such as IBD.

Inputs to the system may also include other drug information, such as disease to be treated, class of drugs, route of administration, dose strength available, preferred dosing amount (e.g., 100 mg vial, 50 mg tablets, etc.), and whether the specific drug is fully human or not (e.g., chimeric). The drug information may be used to determine the available treatment options for a patient, the selected model, and the model parameters. For example, patients treated for IBD often have a higher clearance rate than those without IBD, and a drug dosing regimen for a treatment with IBD must be adjusted accordingly. The preferred dosing amount may alter a dosing regimen before the regimen is recommended for a patient. For example, if a drug is only available in 100 mg vials, the recommended dose amount may be rounded to the nearest 100 mg increment. In some implementations, the drug information excludes information identifying the drug currently used to treat the patient. For example, the drug data may be generic to a drug class.

The physiological data is generally indicative of one or more measurements of at least one physiological parameter of the patient. This may include at least one of: medical record information, markers of inflammation, an indicator of drug elimination such as an albumin measurement or a measure of C-reactive protein (CRP), a measure of antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score, a disease activity score (DAS), a Sharp/van der Heijde score, and demographic information.

The target response may be selected by a physician based on his/her assessment of the patient's tolerance and response to drug therapy. In an example, the target response includes a target drug concentration level of a drug in a sample obtained from the patient (such as a concentration maximum, minimum, or exposure window), and may be used to determine when a patient should receive a next dose and an amount of that next dose. The target drug concentration level may include a target drug concentration trough level; a target drug concentration maximum; a target drug area under the concentration time curve (AUC); both a target drug concentration maximum and trough; a target pharmacodynamic endpoint such as blood pressure or clot time; or any suitable metric of drug exposure.

The inputs described above (e.g., the concentration data, the physiological data, drug information, and the target response) are used by the systems and methods of the present disclosure to personalize a dosing regimen recommendation for a patient. Based on the received inputs, the systems and methods described herein set parameter values for a computational model (such as any of the model parameters described in U.S. patent application Ser. No. 15/094,379 (the '379 Application), published as U.S. Patent Application Publication No. 2016-0300037, filed Apr. 8, 2016, and entitled “Systems and Methods for Patient-Specific Dosing”, which is hereby incorporated by reference in its entirety) that generates predictions of concentration time profiles of the drug in the patient. In some implementations, the computational model is a Bayesian model. For example, the computational model may take into account historical and/or present patient data to develop a patient-specific targeted dosing regimen. As discussed in the '379 Application, the computational model may comprise a pharmacokinetic component indicative of a concentration time profile of the drug, and a pharmacodynamic component based on synthesis and degradation rates of a pharmacodynamic marker indicative of the patient's individual response to the drug. The computational model may be selected from a set of computational models that best fits the received physiological data. For example, if a patient is a 45 year old man, the system may select a computational model specific to men between the ages of 30 and 50 years of age. This computational model can be individualized to a specific patient by accounting for patient-specific measurements (such as the additional concentration data and additional physiological parameter data described herein).

In some instances, the systems and methods of the present disclosure determine a dosing regimen recommendation based on how a patient responded to previous treatment methods. As an example, when a patient does not respond to a treatment involving one drug (e.g., a previously administered drug), physicians sometimes switch the patient to a different drug (e.g., a currently administered drug); the drugs may be related to one another (e.g., from the same class of sharing a common mechanism of action). Lack of response to treatment is sometimes referred to as treatment “failure”, which can sometimes occur in patients with high elimination rates. In these patients, the drug is often eliminated from the body before the full beneficial effects of the drug are realized by the body. Generally speaking, an IBD patient with a high elimination rate for one drug (previously-administered) may be expected to also have a high elimination rate for another similar drug (yet to be administered) because the mechanisms of clearance for similar drugs are generally similar as well. In this manner, the elimination rate (as reflected by the measured drug concentration levels) of the previously administered drug may inform the elimination rate of the drug to be administered. The systems and methods described herein may exploit this correlation to use historical patient data involving one drug (e.g., a previously administered drug) in determining a recommended dosing regimen for another drug (e.g., a drug currently used to treat the patient). For example, a doctor may prescribe adalimumab for a patient. The patient may have a higher-than-average rate of clearance for adalimumab. If the patient fails therapy on adalimumab, the doctor may then attempt therapy with infliximab. The systems and methods described herein then take the higher-than-average clearance of adalimumab into account when calculating dosing regimens for infliximab, because the patient is likely to clear infliximab at a higher-than-average rate as well owing to the fact that both adalimumab and infliximab are mAbs with similar clearance mechanisms.

The systems and methods described herein determine and provide recommended dosing regimens using an iterative approach. In an example, an initial dosing regimen (determined based on the patient's available information and the physician's experience) is administered to a patient. Data indicative of the patient's response or reaction to the initial dosing regimen, such as the patient's physiological and/or concentration data, is provided to the system as feedback on the initial dosing regimen. Then, all, some, or none of that data is used as inputs to a computational model that calculates an updated dosing regimen that is provided to the physician as a recommendation. The physician may choose to administer the dosing regimen exactly as recommended, or the physician may choose to slightly alter the recommended dosing regimen before administering it. For example, the recommended dosing regimen may include a specific dosing interval (e.g., 4 weeks) and a specific dose amount (e.g., 1.9 vials). The physician may select to alter the regimen to accommodate the patient's schedule (e.g., if the patient can only return for another dose at 4 weeks and 1 day), to round up to a particular number of vials (e.g. to 2 vials), or both. This iterative approach is described in detail below.

Using the computational model and the set parameters described above, the systems and methods describe herein determine a first pharmaceutical dosing regimen for the patient. The first pharmaceutical dosing regimen comprises at least one dose amount of the drug and a recommended schedule for administering the at least one dose amount of the drug to the patient. The recommended schedule includes a recommended time for administering a next dose of the drug to the patient, such that a predicted concentration time profile or pharmacodynamic marker profile of the drug in the patient in response to the first pharmaceutical dosing regimen is at or above the target drug exposure level at the recommended time. In some implementations, the dosing regimen may be displayed (e.g., through a user interface). In some implementations, a physician may receive or view (e.g., through a user interface) the first dosing regimen. The physician may decide to alter the dosing regimen before it is administered to a patient.

Once the physician has decided on a course of treatment, doses may be administered to the patient and additional data (indicative of how the patient is responding to the doses) is received. In some implementations, after a start of administration of a dosing regimen based at least in part on the first pharmaceutical dosing regimen to the patient, the systems and methods described herein receive additional concentration data and additional physiological data obtained from the patient. A medical professional may choose to administer the dosing regimen to the patient exactly as recommended by the system. However, the medical professional may also choose to alter the first pharmaceutical dosing regimen prior to administration of the drug. For example, the medical professional may choose to round the dose amount up or down, may choose to alter the dosing times to better fit patient and doctor schedules, may make any suitable alteration to the first pharmaceutical dosing regimen, or any suitable combination thereof. For example, the first pharmaceutical dosing regimen may stipulate a dose of 100 mg on April 20 but the physician may decide to instruct administration of 90 mg on April 22. Details of the administered dose(s) can then be input into the system. The system may then receive additional data (e.g., concentration and physiological parameter data) indicative of how the patient is responding to the administered dose(s).

The systems and methods described herein may display information to a user. The system may display information through a user interface that physician or other user can interact with. For example, the user interface may display a dosing regimen (e.g., as shown in FIG. 13 and described below) or may display the concentration and/or physiological parameter data. In some implementations, a user can toggle or otherwise choose which data to view on the user interface. In some implementations, the predicted concentration time profile of the drug in the patient in response to the first pharmaceutical dosing regimen, as generated by the computational model, is displayed. An indication of at least some of the concentration data and the additional concentration data, the physiological data and the additional physiological data, the target drug exposure level, and the recommended time as a point where the predicted concentration time profile of the drug intersects with the target drug exposure level may also be displayed. For example, a physician may use a graphical user interface to input target responses for a patient, input patient data (e.g., physiological and concentration data), and view predicted concentration time profiles for the patient in response to different dosing regimens.

Based on how the patient is responding to the administered dose(s) that were based at least partly on the first dosing regimen, inputs to the system may be wholly or partially excluded to best determine a new dosing regimen for the patient. In some implementations, the inputs to the system or method are updated, based on a health status of the patient, which may be indicated by the physiological data and the additional physiological data. In particular, the physiological data and the additional physiological data may indicate a decline in health of the patient, which may be an abrupt and clinically significant deviation from the patient's prior pattern of behavior (e.g., how the patient was previously reacting to the administered drug) or an expected patient behavior. The systems and methods described herein may receive an indication of a health status of the patient, which may indicate a decline in health of the patient. In some implementations, the decline in health may be determined by a physician, who provides the indication as an input into the system over a user interface. For example, the physician may view the patient's concentration data and physiological parameter data on a display, determine the patient's health status from the viewed data (as well as any other observable data available to the physician), and then click a button on the user interface, or otherwise indicate the patient is not doing well. In this case, the system takes into account the physician's input when selecting which inputs to include to the next iteration of the model, as is described in detail below. Alternatively, the system and methods described herein may automatically determine the patient's health status based on the patient's physiological and/or concentration data, which may indicate a decline in health.

In either case (e.g., whether the physician or the system determines the patient's health status), the health status may be determined by selecting a physiological parameter from the at least one physiological parameter in physiological data and the additional physiological data of the patient; determining a rate of change of the selected physiological parameter of the patient; retrieving a threshold value from a database that correlates rates of change of physiological parameters with threshold values; and determining that the determined rate of change is greater than the retrieved threshold value. Determining the rate of change may comprise identifying, from the physiological data and the additional physiological data, first data indicative of the selected physiological parameter of the patient at a first time and second data indicative of the selected physiological parameter of the patient at a second time; comparing the first data and the second data to determine an amount of change in the selected physiological parameter of the patient; determining a time interval between the first time and the second time; and determining, from the amount of change and the time interval, the rate of change of the selected physiological parameter of the patient. For example, a patient's weight might have been historically holding steady or improving, but the patient's weight may suddenly begin to rapidly decrease and the patient's concentration data no longer matches predicted concentration values. In this situation, the physician may click a button on the user interface indicating the patient is failing. This input pushes the systems and methods of the present disclosure into a specific mode of operation to quickly respond to the patient's sudden decline in health (e.g., by altering the inputs that the computational model considers when calculating a dosing regimen).

In some implementations, the inputs are updated to wholly or partially remove the concentration data from the inputs and include the additional physiological data in the inputs. In particular, in some implementations, the updated inputs may include the physiological data, the additional physiological data, and the target drug exposure level, and may exclude the concentration data and the additional concentration data. In this case, the system essentially ignores the concentration data and instead uses the physiological parameter data as a basis for the dosing regimen calculation. In some implementations, updating the inputs is further based on the additional concentration data being consistent with the concentration data, meaning the system double checks that the concentration data is consistent (e.g., that it somewhat matches predicted concentration values and is not suddenly far off from the prior concentration data).

In one example, it may be desirable to exclude the entirety of the concentration data in an iteration of the model when the target response is not appropriate. In this case, a patient may have concentrations of a drug in their system that matches the target response (and are steady and therefore are not rapidly changing), but his/her physiological parameters may indicate he/she is not responding to the drug. For example, his/her weight may be rapidly decreasing, his/her clearance may be increasing, or there may be any other suitable change in a physiological metric that indicates a poor response or lack or response to the drug. This situation (where concentration data indicates the patient has the appropriate exposure to the drug but physiological data indicates the patient is not doing well) indicates that the target response for the patient was incorrect or otherwise not suitable for the patient. In some instances, the decline in health is a clinical judgement, meaning a medical professional decides the patient's health status or at which point the patient is not responding to the dosing regimen. Once the medical professional makes this determination, he/she can “turn off” the concentration data so that the concentration data is wholly excluded from the next iteration of the model. The systems and methods described herein may receive an indication of this decline in health, for example, through a key click, toggle button, or mouse indication input by the medical professional. The systems and methods described herein would then proceed to determine a second pharmaceutical dosing regimen based on the patient's physiological parameters, and not on the patient's concentration data. Once the patient is stable and responding to the drug (e.g., when the patient's physiological parameters are acceptable or the patient's health status is acceptable), the physician can adjust the target response.

Sometimes, rather than excluding the entirety of the concentration data, it is desirable to only exclude a portion of the concentration data, and include a remainder of the concentration data in the inputs to the model. This situation may arise if a patient takes a “sudden turn for the worse.” The sudden turn for the worse may be indicated by the concentration data and/or the physiological parameter data. In this case, the concentration data is initially updated to include both the original concentration data as well as the additional concentration data (e.g., the most recent concentration data point). If the patient has experienced a sudden or unexpected material change to their concentration data (as determined by the patient's physician, for example), the system may select to exclude the “older” concentration data and include the more recent concentration data (such as the most recent 1, 2, 3, 4, 5, 6, or any other suitable number of data points, or the most recent 10%, 20%, 30%, 40%, 50%, or any other suitable percent of the concentration data) in the next iteration of the model. In this manner, the concentration data is split into two portions: the more recent data that is included (which may be referred to herein as a subset of the concentration data) and the older data that is excluded (which may be referred to herein as a remaining portion of the concentration data). For example, the drug concentration level in a patient may be measured 10 times, such that the aggregate concentration data includes 10 data points. These 10 data points may be divided such that the subset includes the most recent two data points (to be included in the next iteration of the model), while the remaining portion includes the oldest 8 data points (to be excluded from the next iteration of the model). The excluded data will not be used in the computation and determination of the updated dosing regimen, but the included data will be used. In this case, because the subset consists of only two out of the total ten measurements, the two remaining data points will be “weighted” more heavily in the system's calculations than they would have been if all ten data points were included. Thus the system relies more heavily on recently measured concentration data than it would have if all ten data points were taken into account. In this case, only considering the most recent concentration data (and excluding older concentration data) in an iteration of the model is desirable because this allows the system to focus solely on the recent concentration data that may indicate the patient's “sudden turn for the worse” change in health status. Focusing solely on that data, and ignoring the historical concentration data that may indicate a normal health status for the patient, allows the system to provide a recommended dosing regimen that more effectively targets the patient's current health status.

In some implementations, the physician determines that there has been a material change in the patient's concentration data, and provides an input to the system indicating that there has been a material change. This input may be provided via a key click, toggle button, or mouse indication input on the user interface. Additionally or alternatively, the physician provides an input indicating to the system exactly which concentration data points to keep for the next iteration of the model, and/or exactly which concentration data points to exclude for the next iteration of the model. This way, the physician may set the model inputs based on the concentration data points that the physician wishes to be emphasized in the next recommended dosing regimen. In this case, the system does not itself perform any calculation or analysis to select which concentration data points to include or exclude for a particular iteration of the model, but rather determines these inputs based on what is received from the physician.

In some implementations, the systems and methods described herein determine whether there is a material change in concentration based on whether response data (e.g., the concentration data) is representative of an abrupt and clinically significant deviation from prior pattern of behavior. For example, a patient's drug exposure may be measured 10 times. In this example, the first nine measurements the measured drug concentration matched the predicted drug concentration to within 20%, but on the last measurement (the tenth measurement), the measured concentration differed from the predicted concentration by 50%. This sudden deviation from the prior pattern of behavior may indicate a change in the patient's ability to clear the drug or a change in the patient's health. In cases where a drug concentration in a patient suddenly changes, the inputs to the model are updated to respond quickly to the patient's changing health, by removing either a portion of the concentration data or the entire set of concentration data from the model inputs.

In an example, a patient takes a sudden turn for the worse. His/her last concentration data point is at odds with previous concentration data and, his/her health may be rapidly decreasing (e.g., as evidenced by declining physiological parameters). While this sort of rapid decline in patient health is common early in treatment, it can happen at any time during drug therapy. A medical professional may view the patient's concentration and/or physiological parameter data and determine that the patient is taking a turn for the worse. In response, the medical professional may click a button, press a key, or otherwise indicate that amount of concentration data should be limited. In some examples, this process may be automated such that the system determines whether a patient experiences a material change in concentration. In response to an indication that the patient is taking a turn for the worse (e.g., experiencing a sudden or material change in concentration), the number of concentration data points used when calculating the second pharmaceutical dosing regimen may be limited to the recent concentration data points, allowing them to be weighted more heavily in the calculation of the second pharmaceutical dosing regimen.

Often, when a patient is early in treatment, there is not much patient data. In particular, historical data indicative of how the patient has responded or reacted to different doses of the medication is typically unavailable. In this case, when there is little patient data to rely on, the systems and methods of the present disclosure may limit the number of concentration data points considered in an iteration of the model to just a single data point, which may be the most recent data point. The most recent data point may be the sole concentration data input to the model because that data point most accurately reflects the current status of the patient, and allows the systems and methods of the present disclosure to heavily weight that data point in the determination of a recommended dosing regimen. In this manner, the inputs to the model are determined based on whether an administered period of treatment of the first pharmaceutical dosing regimen is greater than a proportion of a total length of time of the first pharmaceutical dosing regimen (e.g., whether the patient is early in treatment or not).

The systems and methods of the present disclosure generally relate to excluding a certain type or a portion of a certain type of data, in determining a recommended dosing regimen. In order to determine whether to exclude all of the concentration data or just a portion of the concentration data for a specific patient, the systems and methods of the present disclosure may first determine how the patient is reacting physically (e.g., by analyzing the physiological parameter data for the specific patient) to a particular treatment, and/or whether there has been a sudden material change in the patient's concentration data. In one example, if the physiological parameter data for a patient is indicative of a decline in health for that patient, then the concentration data that is included in the next iteration of the model may consist of the most recent 1, 2, or 3 data points (or any other suitable number of data points). In another example, if there is a sudden change in the concentration data for the patient and the patient's health status is not acceptable (as indicated by the physiological parameters and/or by a physician's input to the system), then the most recent concentration data points may be used (to the exclusion of the older concentration data points) as input to the next model iteration. Accordingly, the systems and methods described herein may limit the number of concentration data points considered in the determination of the second pharmaceutical dosing regimen.

Sometimes, the value provided to the system for a particular data point, such as a concentration data point, is an error. For example, the user providing the input to the system may accidentally execute a typographical error, such that the most recently entered input has suddenly and dramatically departed from expected values. In this case, when an anomalous concentration data point value is provided, but the patient's health status, as indicated by the physiological parameter data, is unchanged or otherwise stable and acceptable, the present disclosure provides a check for whether the latest concentration data point value was provided in error. In an example, a clinician may be prompted to confirm the most recently entered value (which may be referred to herein as the additional concentration data), or the system may be configured to automatically determine that the value is an anomaly, if it falls outside a certain range that may be predetermined or set according to the patient's historical concentration data. If the value is an anomaly, the value is permanently removed from the concentration data and is not included in the model inputs for any of the next iterations of the model. The present disclosure may perform this check for each data point that is entered into the system. Any data that is determined to be not anomalous (either determined by the system or by the user) is included in the next model iteration.

### BRIEF DESCRIPTION OF THE DRAWINGS

FIG. 1 shows a flowchart for modifying adaptive dosing systems, according to an illustrative implementation;

FIG. 2 shows a system diagram of a computer network for adaptive dosing systems, according to an illustrative implementation;

FIG. 3 shows a process for modifying adaptive dosing systems, according to an illustrative implementation;

FIG. 4 shows an example display of a user interface on a clinical portal that provides a graph of predicted concentration time profiles and recommended dosing regimens for a specific patient, Patient A, according to an illustrative implementation;

FIG. 5 shows a plot of drug concentration over time after the dosing regimen shown in FIG. 4 has been modified, according to an illustrative implementation;

FIG. 6 shows a plot of Patient A's drug clearance over time, according to an illustrative implementation;

FIG. 7 shows a graph of a predicted concentration time profile for a specific patient, Patient B, based on a subset of concentration data and physiological parameter data, according to an illustrative implementation;

FIG. 8 shows a graph of a predicted concentration time profile for a specific patient, Patient B, based on physiological parameter data, according to an illustrative implementation;

FIG. 9 shows an example display of a user interface on a clinical portal that provides a graph of a predicted concentration time profile for a specific patient, Patient C, according to an illustrative implementation;

FIG. 10 shows a graph of Patient C's weight over time, according to an illustrative implementation;

FIG. 11 shows a plot of Patient C's C-reactive protein over time, according to an illustrative implementation;

FIG. 12 shows a graph of a predicted concentration time profile for Patient C, based on physiological parameter data, according to an illustrative implementation;

FIGS. 13A and 13B are example displays of a user interface on a clinical portal that provide several recommended dosing regimens, according to an illustrative implementation;

FIG. 14 shows a system diagram of an exemplary computer system, according to an illustrative implementation; and

FIG. 15 shows a process for modifying adaptive dosing systems, according to an illustrative implementation.

### DETAILED DESCRIPTION

Described herein are medical treatment analysis and recommendation systems and methods that provide a tailored approach to analyzing patient measurements and to generating recommendations that are responsive to a patient's specific response to a treatment plan. To provide an overall understanding, certain illustrative implementations will now be described, including a system for predicting a patient's response to a treatment plan and providing and modifying a patient-specific dosing regimen. However, it will be understood by one of ordinary skill in the art that the systems and methods described herein may be adapted and modified as is appropriate for the application being addressed and may be employed in other suitable applications, and that such other additions and modifications do not depart from the scope thereof.

In some computerized dosing recommendation systems utilizing a mathematical model to provide recommended dosing regimens, all known prior data is utilized in the mathematical model. However with some other approaches, such as a Bayesian approach, the model may ignore certain data that is inconsistent with the prior data or that is inconsistent with the patient's past history. For example, if a patient's condition suddenly worsens more quickly than the system can account for, the system may ignore the latest data, which can result in the system recommending an inappropriate dosing regimen. In severely ill patients, physicians often do not have time to collect more data and reassess the patient's condition. The present disclosure provides a computerized dosing recommendation system that systematically accounts for sudden and/or material changes in patients and provides recommended dosing regimens that are expected to improve the health status for patients with rapidly declining health.

The present disclosure provides systems and methods for determining a patient-specific pharmaceutical dosing regimen for a patient, using a computerized pharmaceutical dosing regimen recommendation system. In particular, the systems and methods described herein involve tailoring the inputs to a computational model, based on whether a patient is responding to treatment, has a sudden decline in health, and/or is early in treatment. Conceptually, a prescribing physician is provided with access, in a direct way, to mathematical models of observed patient responses to a medication or class of medications when prescribing the medication to a specific patient. In providing one or more recommended treatment plans for a patient, the mathematical model is used to predict a specific patient's response as a function of observed responses, including medication concentrations, of the medication being used to treat the patient and/or other medications in the class of medications, in the patient's blood and physiological parameters that the model accounts for as patient factors. Accordingly, the system customizes the computational model for a specific patient and the prescribing physician is able to leverage the model in developing a tailored treatment plan for a specific patient.

A dosing regimen (also referred to as a treatment plan) may include a schedule for dosing, one or more dosing amounts, and/or one or more routes of administration. Dosing regimens are not limited to just one drug, but can include multiple drugs, with the same or different routes of administration. A drug (also referred to as a pharmaceutical, medicine, medication, biologic, compound, treatment, therapy, or any other similar term) is a substance which has a physiological effect when introduced into a body.

In some implementations, the systems described herein may not be specific to a particular drug but instead apply to a class, or subset or grouping of drugs used in a drug-agnostic model. As used herein, the term “drug” may refer to a single drug or a class or set of drugs. A class of drugs is a group of drugs larger than one, which exhibit at least one similar pharmacokinetic (PK) and/or pharmacodynamic (PD) behavior, share a common mechanism of action, or a combination thereof. As an example, a set of drugs may treat the same disease or be used for the same indication, examples of which include general inflammatory disease, inflammatory bowel disease (IBD), ulcerative colitis, Crohn's disease, rheumatoid arthritis, ankylosing spondylitis, psoriatic arthritis, psoriasis, asthma, or multiple sclerosis. A set of drugs may have a similar chemical structure. For example, a set of drugs could include monoclonal antibodies (mAbs), anti-inflammatory compounds, corticosteroids, immunomodulators, antibiotics or biologic therapies. A user such as a doctor, clinician, or a user building a drug-agnostic model may define a class of drugs based on specific criteria, and members of that class may be electronically designated in a database as being part of that class. That database is accessible to systems and methods disclosed herein, for use in determining a class-based dosing regimen that could be used for any drug in the class. In some implementations, a dosing regimen output from the model may not be specific to a single drug but may be generic to the class of drugs, and suitable for any drug in that class. For example, a dosing regimen may include a drug-agnostic unit measurement (e.g., one unit, two units, three units, etc, where a unit corresponds to a specified amount of an active agent) and a time or times for administration.

The development and application of drug-agnostic models allow greater utility for a single model. Rather than implementing multiple models, where each model corresponds to a single drug in a class, a drug-agnostic model is applicable to all drugs within the class and may accordingly be applied to a wider range of patients than single-drug models and across a range of administration routes. For example, the models described herein may include a pharmacokinetic drug-agnostic model capable of being used for all biologics used in the treatment of inflammatory diseases. Such a model can be used to propose dose regimens for fully human monoclonal antibodies (mAb), chimeric mAbs, fusion proteins, and mAb fragments (i.e., a range of drugs with differing pharmacokinetic properties but other similarities such as molecular weight and indication) using the same model. The model can be used in a broad patient population, including inflammatory bowel disease, rheumatoid arthritis, psoriatic arthritis, psoriasis, multiple sclerosis, and other such diseases that arise from immune dysregulation. The development and application of drug-agnostic Bayesian models for agents in other broad drug sets (e.g., the aminoglycoside antibiotics, chemotherapeutic agents that cause low white cell counts, etc.) is similarly feasible. Drugs within the class may be administered through a variety of routes, such as subcutaneously, intravenously, or orally. Drug-agnostic models may account for route of administration by taking the route of administration as a variable input to the system, allowing greater flexibility for the model.

Because the drug-agnostic model applies to a set of drugs, rather than only a single drug, the model may retain patient-specific information when a patient is treated with multiple drugs within the set of drugs. For example, the set of drugs may include infliximab, vedolizumab, adalimumab, and other anti-inflammatory biologics. If a patient is treated with one drug (e.g. infliximab), then later treated with another drug (e.g. vedolizumab), the model may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the drug-agnostic model to accurately anticipate the patient's ability to process a drug and thereby provide more suitable, patient-specific dosing regimens when a patient changes drug therapy.

Many of the implementations described herein relate to the treatment of IBD, such as ulcerative colitis or Crohn's disease. Although there is no standard treatment regimen for IBD, the following groups of drugs can be used to treat IBD patients: anti-inflammatory compounds, corticosteroids, immunomodulators, antibiotics or biologic therapies. One recently developed treatment includes biologic therapies (e.g., monoclonal antibodies (mAbs) such as infliximab), which target and bind to an inflammatory protein called tumor necrosis factor (TNF), rendering it inactive. In some instances, a combination of anti-TNF agents, such as infliximab, can be combined with one or more immunomodulatory agents, such as thiopurines. Such combination therapies may effectively lower elimination rates (thereby increasing drug concentration levels in a patient's blood) and reduce formation of anti-drug antibodies.

The biggest challenge in treating a patient with IBD is ensuring that the patient receives adequate exposure to the treatment. The body presents several routes of “clearance” for the drugs. For example, a patient's metabolism may break down-mAbs by proteolysis (breaking down of proteins), by cellular uptake, and by additional atypical clearance mechanisms associated with IBD. For example, due to the nature of the disease, patients with conditions such as focal segmental glomerulosclerosis (FSGS) often suffer from excessive losses of the drug into the urinary and or gastrointestinal tracts. Moreover, in severe IBD, mAbs are sometimes lost in feces through ulcerated and denuded mucosa, creating an additional route of clearance. Overall, IBD patients are estimated to have an infliximab elimination rate that is 40% to 50% higher than other inflammatory diseases, making IBD especially difficult to treat.

The systems and methods described herein may also develop dosing regimens to treat rheumatoid arthritis, psoriatic arthritis, ankylosing spondylitis, plaque psoriasis, low levels of clotting factor VIII, hemophilia, schizophrenia, bipolar disorder, depression, bipolar disorder, infectious diseases, cancer, seizures, transplants, or any other suitable affliction.

The systems and methods described herein may be used to develop dosing regimens any drugs that have elimination rates that would be suitable for use with the models described herein. For example, the systems and methods described herein may be used to develop dosing regimens for any mAbs, aminoglycoside antibiotics, recombinant human factor VIII, infliximab, busulfan, cyclosporine, tacrolimus, mycophenolate, anti-thymocyte globulin, valproic acid, amikacin, gentamicin, methotrexate, tobramycin, vancomycin, warfarin, itraconazole, fluconazole neonatal, fludarabine, carboplatin, imatinib mesylate, velcade, fludarabine, enoxaparin, dabigatran, digoxin, amikacin, gentamicin, meropenem, piperacillin, teicoplanin, tobramycin, vancomycin, voriconazole, flucloxacillin, itraconazole, linezolid, risperidone, various other forms of chemotherapy, antidepressants, antipsychotics, psychostimulants, antidiabetic agents, antibiotics, anticoagulants, anticonvulsants, analgesics, and any other suitable treatment.

Many of the examples described herein are in relation to the pharmaceutical infliximab. However, the implementations described herein may apply to immunosuppressive, anti-inflammatory, antibiotic, anti-microbial, chemotherapy, anti-coagulant, pro-coagulant, or any other suitable drug.

Patient-specific drug dosing regimens may be provided as a function of mathematical models (e.g., pharmacokinetic and/or pharmacodynamic models) that are updated to account for observed patient responses. This is described in detail in the '379 Application, which is incorporated herein by reference in its entirety. In particular, a specific patient's observed response to an initial dosing regimen is used to adjust the dosing regimen. The patient's observed response (e.g., an observed drug concentration in the patient's blood) is used in conjunction with the mathematical model and patient-specific characteristics to account for between-subject-variability (BSV) that cannot be accounted for by the mathematical model alone. The observed responses of the specific patient can be used to refine the models and related forecasts, to effectively personalize the models so that they may be used to forecast expected responses to proposed dosing regimens more accurately for a specific patient. In this manner, observed patient-specific response data is effectively used as “feedback” to adapt a generic model describing typical patient response to a patient-specific model capable of accurately forecasting a patient-specific response, such that a patient-specific dosing regimen can be predicted, proposed and/or evaluated on a patient-specific basis. Using the observed response data to personalize the models allows the models to be modified to account for BSV that is not accounted for in previous mathematical models, which described only typical responses for a patient population, or a “typical for covariates” response for a typical patient having certain characteristics accounted for as covariates in the model.

Bayesian analysis may be used to determine a recommended dosing regimen. This is described in detail in U.S. patent application Ser. No. 14/047,545, filed Oct. 7, 2013 and entitled “System and method for providing patient-specific dosing as a function of mathematical models updated to account for an observed patient response” (“the '545 Application”), which is incorporated herein by reference in its entirety. As is described in the '545 Application, a Bayesian analysis may be used to determine an appropriate dose needed to achieve a desirable result, such as maintaining a drug's concentration in the patient's blood near a particular level. In particular, the Bayesian analysis may involve Bayesian averaging, Bayesian forecasting, and Bayesian updating, which are described in detail in the '545 Application.

Bayesian systems are particularly robust to data entry errors. If a new data entry (e.g., corresponding to an observation related to the patient, such as an observed new state of the patient) is substantially different from past data (e.g., previous data entries), the Bayesian system may ignore the new data entry until it receives further data confirming this new state. However, in an emergency clinical setting, when a patient's health is rapidly declining, medical professionals often do not have time to wait for such a system to confirm this change in state. For example, a patient may be in sepsis. A patient may be responding well, but the patient's kidneys may suddenly fail. Stopping treatment, or continuing a previous treatment without modification, may cause the patient to fail the drug therapy, or even harm or kill the patient. Instead, the patient's dosing regimen must be quickly adjusted to account for the kidney failure. Additionally, at any time in therapy patients can develop anti-drug antibodies which if dealt with quickly can sometimes be reversed. The present disclosure describes several ways to remedy such problems.

The systems and methods described herein differ from (and are counterintuitive to) typical Bayesian systems at least because some data is specifically and intentionally excluded from dosing regimen calculations. For example, if a patient takes a sudden and unexpected turn for the worse and/or the patient's disease is progressing faster than would be expected, the systems and methods described herein may exclude older concentration data or exclude concentration data from the calculation entirely. In this way, the systems and methods described herein can give more weight and place more importance on more recent measurements-thereby increasing the ability to react quickly to a patient health downturn.

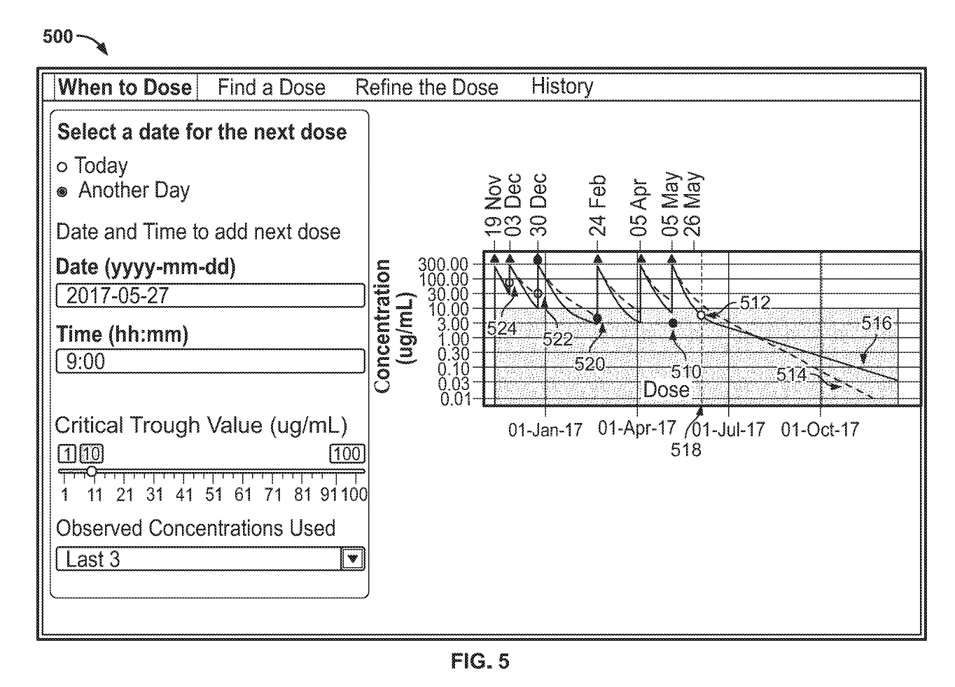

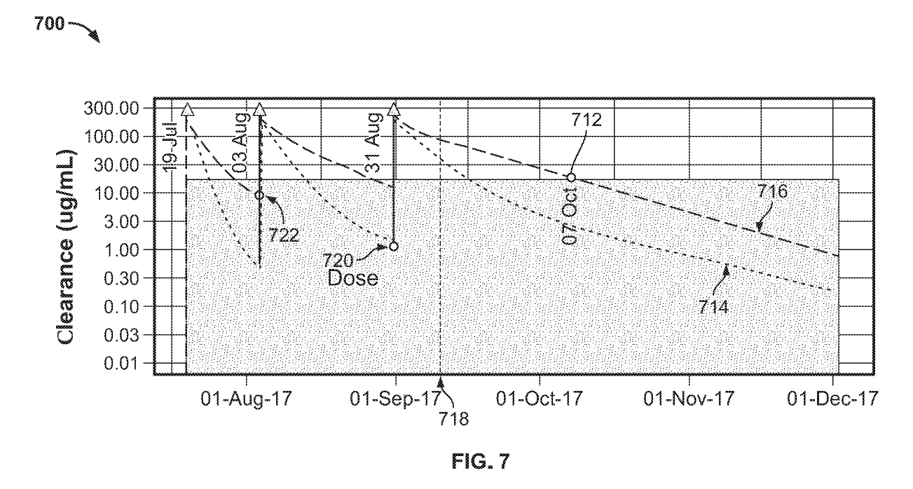

One way to account for a rapid change in patient status is to truncate the number of data entries used by the system to include only the recent observations (e.g., the observed concentration levels of a patient), or to otherwise weight these more recent observations more heavily than older observations. Examples of truncating the number of data entries used by the system are described in relation to FIGS. 5 and 7.

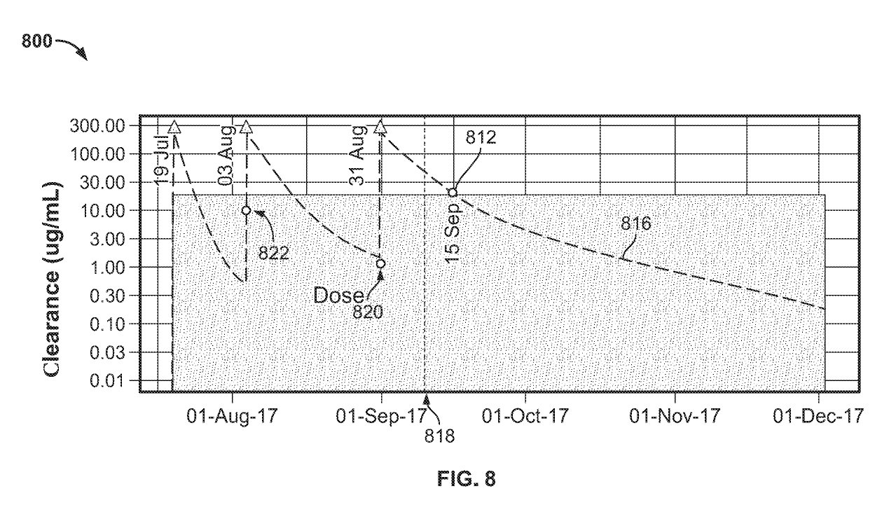

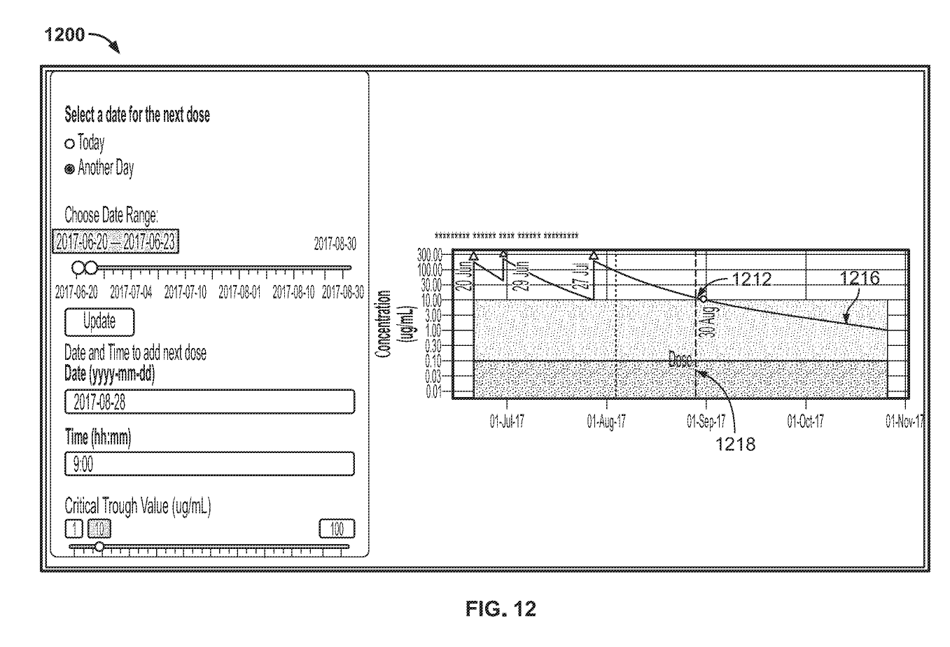

Another way to account for a rapid decline in patient health is to exclude all of the observed concentration data from the data considered by the system. Examples of excluding all observed concentration data from the data considered by the system are described in relation to FIGS. 8 and 12. In some cases, a patient reaches the target response (e.g., a drug exposure level such as a target concentration trough level) but his/her health is still declining and he/she is failing to respond to the drug. This may indicate the target response was incorrectly chosen. To quickly readjust the patient's dosing regimen, and effectively “buy time” for a physician to recalculate an appropriate target response, all of the observed concentration data is then excluded, rather than only a subset of the concentration data. By completely excluding the observed concentration data from the data considered by the system, the system is able to react quickly to the changing health state of the patient. At a later time, once the patient is stable and no longer worsening rapidly, a new target response may be entered or calculated and the system inputs may be adjusted to include the concentration data, or a portion thereof.

Early in therapy, a patient's health can worsen rapidly. For example, patients are more likely to fail early in therapy (during the induction phase of treatment) than later in therapy (during the maintenance phase of the treatment). For example, the drug infliximab is sometimes used to treat a patient with inflammatory bowel disease (IBD). Types of IBD may include ulcerative colitis and Crohn's disease. During the induction phase of treatment, which lasts about 3 months for infliximab (e.g., in treating IBD, rheumatoid arthritis, psoriatic arthritis, ankylosing spondylitis, plaque psoriasis, or any other suitable affliction), the patient is at his/her sickest. Later, during the maintenance phase of treatment, the patient has already been through the most vulnerable stage of treatment, and the administration of infliximab is done to maintain the patient's health status. Additionally, at any time in therapy, patients can develop anti-drug antibodies, which essentially inactivate the therapeutic effects of the treatment and can even introduce their own adverse effects on the patient's body. If the anti-drug antibodies can be responded to quickly, in some cases it may be possible to reduce or eliminate the antidrug antibodies. If only limited data is available, truncating or restricting the time span of data to use for updating the model does not greatly improve the model's ability to provide appropriate dose recommendations. In an example, the specific patient may have an actual response to the treatment that falls within the range of the target response. However, the patient may fail to respond to the therapy (as indicated by the patient's physiological parameter data, for example). In this case, rather than selecting only a subset of the observed concentration data to include for consideration in the model, all of the observed concentration data may be excluded. By completely excluding the observed concentration data from the data considered by the system, the next iteration of the model would provide a recommended dosing regimen that is based solely on the patient's physiological parameter data (including the patient's lab data, for example), regardless of the patient's actual drug exposure. In this manner, the system is able to react quickly to the realization that the patient is not responding to treatment in a desirable manner. At a later time, once the patient is stable and no longer worsening rapidly, the system inputs may be adjusted to include the concentration data, or at least a portion thereof.

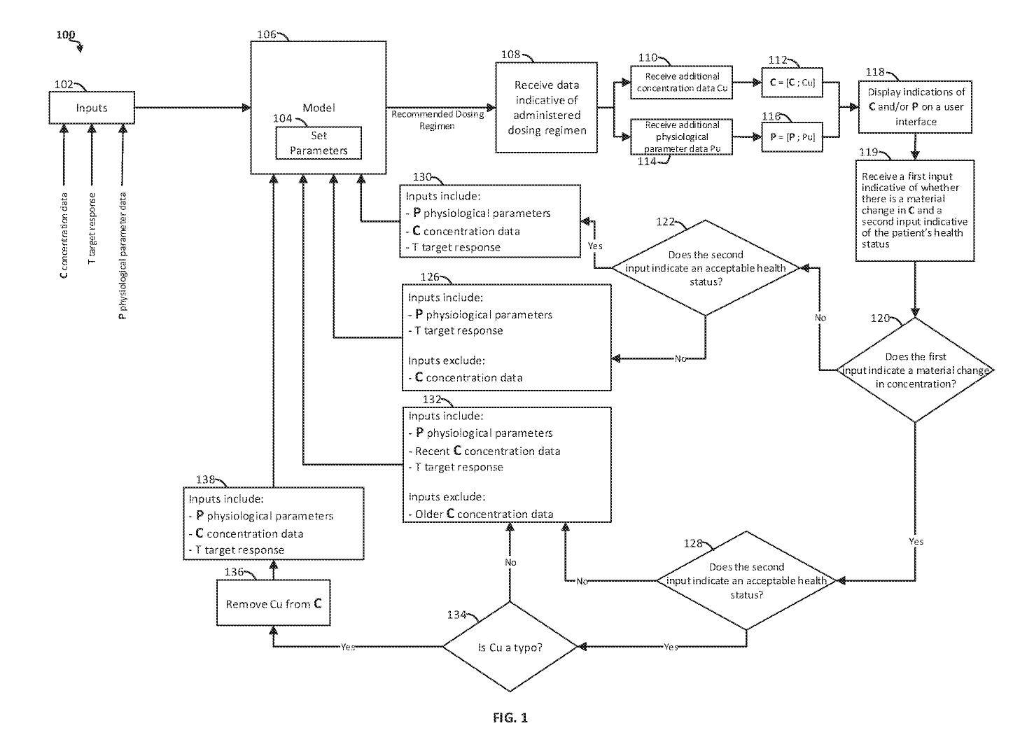

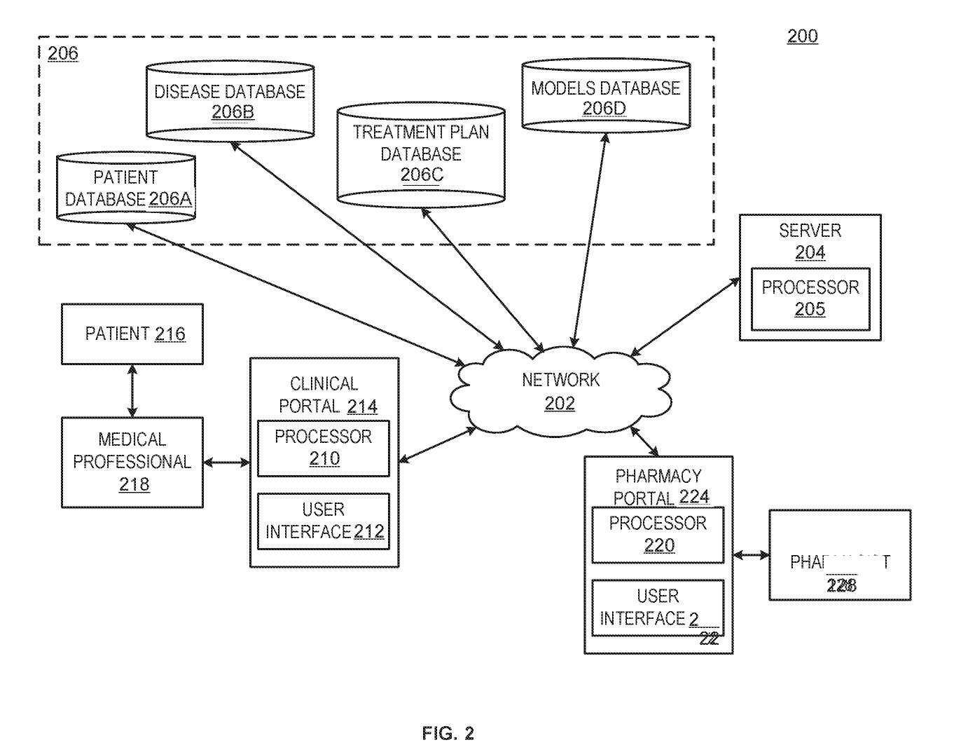

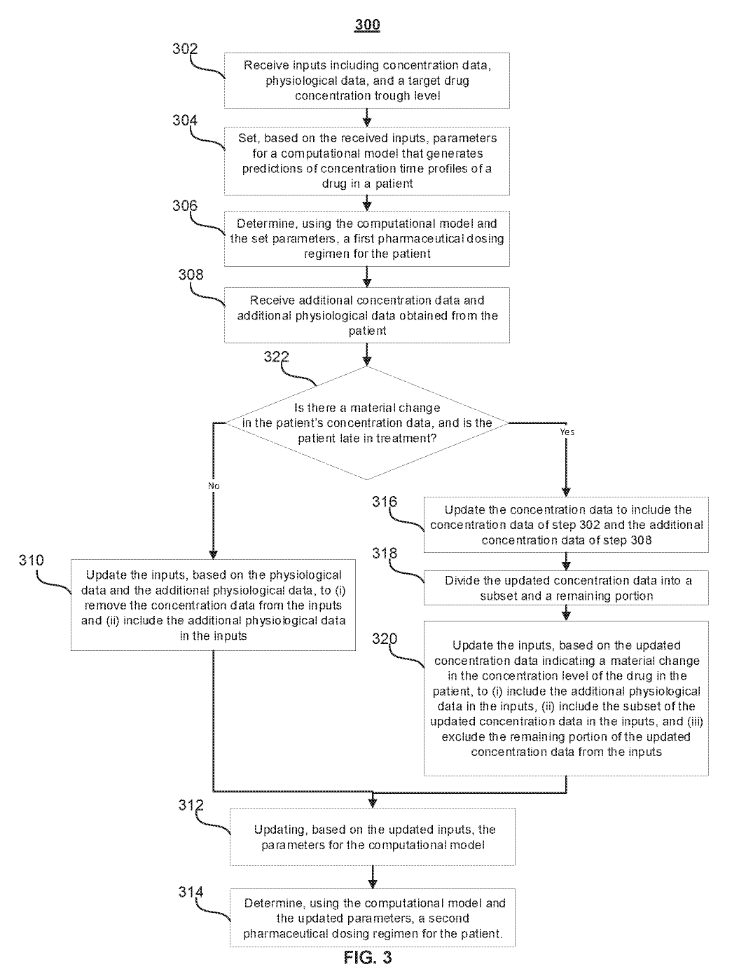

FIG. 1 shows a flowchart 100 implemented by a system (such as system 200 described in relation to FIG. 2, for example, or any of the components of the system 200, such as the server 204) or a method (such as the process 300 described in relation to FIG. 3, for example) for modifying adaptive dosing systems, according to an illustrative implementation. As described above, traditional dosing systems may automatically treat data that suddenly changes data as an error or as an anomaly and may select to ignore that data. This presents a problem for patients experiencing a sudden or substantial “turn for the worse.” In such situations, it is paramount to use dosing regimens that are capable of taking into account a patient's rapidly worsening condition. FIG. 1 depicts multiple situations and solutions when treating a patient with an adaptive dosing system.

FIG. 1 is representative of five scenarios, each of which indicates a different method of setting the inputs and parameters of the mathematical model used to provide a recommended dosing regimen for patients with different statuses. All scenarios begin after block 102, at which the inputs (e.g., the concentration data, target trough level, and physiological parameter data) to the system are set. Each scenario begins and ends at block 106, and includes at least blocks 106, 108, 110 and/or 114, 112 and/or 116, 118, 119, and 120. Depending on the patient's status (as evaluated by whether there are material changes in concentration data, as determined at decision block 120) or whether the patient's physiological parameters are acceptable or not (as determined at decision blocks 122 or 128), the system executes one of five different scenarios. The description below begins with a description of block 102, followed by a brief description of each of the five scenarios, and a more detailed description of the blocks involved in each scenario.

At block 102, the system receives inputs for a specific patient. The inputs include concentration data \vec{C}, a target trough T, and physiological parameter data \vec{P}. These inputs correspond to the inputs of step 302 of FIG. 3, as described below.

Concentration data \vec{C} is indicative of at least one concentration level of a drug or class of drugs, or an analyte related to a drug or class of drugs in one or more samples obtained from the patient. The samples may include blood, blood plasma, urine, hair, saliva, or any other suitable patient sample. For example, a medical professional may draw a sample of blood from the patient and may measure a concentration of a biomarker in the blood sample to determine a concentration of the drug in the patient's blood. Concentration data \vec{C} can be a single data point, or a vector or matrix of any number of data points. For example, \vec{C} may represent one biomarker in blood and another biomarker in urine, or two different biomarkers in blood.

Target response T may be selected by a physician based on his/her assessment of the patient's pain tolerance and response to drug therapy. The target response may be a quantifiable measure, such as a drug concentration level or drug exposure level, which may include a target drug concentration trough level; a target drug concentration maximum; both a target drug concentration maximum and trough (defining a target window or range); a target pharmacodynamic endpoint such as blood pressure or clot time; or any suitable metric of drug exposure. The drug concentration levels and troughs described herein may refer to a single drug or a class of drugs. In one example, the target response defines a critical trough value corresponding to a threshold concentration level, where it is undesirable for the patient's concentration to be below the critical trough value. For example, the target trough T may be 10 ug/mL for infliximab. The systems and methods of the present disclosure may be configured to provide dosing regimen recommendations such that the concentration data in the patient is not predicted to fall substantially below this target trough at any point in time, but instead approaches the target trough value at the time the next dose is administered to the patient. In another example, when the target response T is a target maximum value, it may be desirable to provide dosing regimen recommendations such that the concentration data in the patient is not predicted to rise substantially above this target maximum at any point in time. When the target response T defines a target window, including a critical trough value and a critical maximum value, the dosing regimen recommendations may be provided such that the concentration data in the patient is not predicted to fall substantially below or above the target window.

Physiological parameter data \vec{P} can include any number of measurements in relation to a patient's health. For example, physiological parameter data may include medical record information and may be indicative of a measurement of at least one of markers of inflammation, an albumin measurement, an indicator of drug clearance, a measure of C-reactive protein (CRP), a measure of antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score a disease activity score (DAS), a Sharp/van der Heijde score, and demographic information. Physiological parameter data \vec{P} can be a single data point, or a vector or matrix of any number of data points relating to any number of physiological parameters. For example, the physiological parameter data may comprise markers relating to CRP, weight, and clearance, each at three different points in time.

Scenario 1 includes blocks 106, 108, 110 and/or 114, 112 and/or 116, 118, 119, 120, 122, and 130 before returning to block 106. Scenario 1 represents a patient who is reacting well to therapy (e.g., has an acceptable health status, as indicated by the patient's physiological parameters, for example, and is not experiencing a material change in concentration). In Scenario 1, no concentration data is excluded from the next iteration of the model. Scenario 1 therefore represents an operating procedure for a patient who is reacting well to treatment. For example, a patient with inflammatory bowel disease (IBD) may be reacting well (meaning the concentration data values are as expected and the physiological parameters are not changing negatively) to treatment with a drug (e.g., infliximab).

Scenario 2 includes blocks 106, 108, 110 and/or 114, 112 and/or 116, 118, 119, 120, 122, and 126 before returning to block 106. In Scenario 2, all concentration data is excluded from the inputs to the next iteration of the model. Scenario 2 occurs when a patient's concentration data seems appropriate and has not suddenly changed (e.g., as determined at decision block 120), but the patient's health status is not acceptable (e.g., as determined at decision block 122). For example, the observed concentration data may be in general alignment with predictions provided by the model, but clinically the patient is getting worse (as indicated by the physiological parameter data, such as losing weight, increasing CRP, clearance, disease severity, anti-drug antibodies, albumin). In this case, the selected target response may be inappropriate and may need to be reset. Accordingly, completely removing all of the concentration data from the next iteration in Scenario 2 allows the next recommended dosing regimen to rapidly account for the physiological changes in the patient, without regard to the “normal-looking” concentration data. In this manner, the physician may react quickly to an apparently failing patient, while allowing the physician to optionally update the target response.

Scenario 3 includes blocks 106, 108, 110 and/or 114, 112 and/or 116, 118, 119, 120, 128, and 132 before returning to block 106. Scenario 3 occurs when a patient experiences a material change in concentration (e.g., as indicated at decision block 120), and the patient's health status is not acceptable (e.g., as indicated at decision block 128). Both of these indications are signs that the patient has taken a sudden turn for the worse. For example, the patient may be undergoing treatment for sepsis and suddenly develop kidney failure. In Scenario 3, a subset of the concentration data (e.g., an older portion of the concentration data) is excluded from the model inputs for the next iteration, leaving only the recent concentration data to be included as input concentration data in the system's dosing regimen calculation (as set at block 132). This setting of the inputs to the next iteration of the model ensures the system considers only the most recent concentration data to quickly react to the substantial turn for the worse in patient health, and further ensures the most recent concentration data point is not considered by the system to be an error. In some implementations, when the system excludes the older concentration data \vec{C}, on future iterations of the system (e.g., through any of Scenarios 1 through 5) the concentration data vector \vec{C} may start as an empty vector, meaning that only new concentration data measurements Cu (e.g., the most recent concentration data point received at step 110) will be included as inputs for dosing regimen recommendation calculations. In some implementations, the physician may indicate which, if any, of the concentration data \vec{C} should be excluded or included in future iterations of the system (e.g., through any of Scenarios 1 through 5).

Scenario 4 includes blocks 106, 108, 110 and/or 114, 112 and/or 116, 118, 119, 120, 128, 134, and 132 before returning to block 106. Scenario 4 occurs when the patient experiences a material change in concentration (e.g., determined at decision block 120) but the patient's health status is acceptable (e.g., determined at decision block 128), and the additional concentration data Cu (e.g., the most recent concentration data point received at step 110) is not in error (e.g., determined at decision block 134). For example, the patient may be undergoing treatment for IBD. The patient's weight and CRP levels may be fine or improving, but the concentration level of the infliximab in the patient's blood may dramatically decrease. In Scenario 4, a subset of the concentration data is excluded from the inputs to the model at block 132 (similar to Scenario 3). If the patient is early in treatment (e.g., the patient has received a small number of doses of infliximab), the system may limit the concentration data to the single most recent data point (e.g., Cu).

Scenario 5 includes blocks 106, 108, 110 and/or 114, 112 and/or 116, 118, 119, 120, 128, 134, 136, and 138 before returning to block 106. In Scenario 5, the system determines that while there was a material change in concentration (e.g., determined at decision block 120), the patient's health status is acceptable (e.g., determined at decision block 128) and the additional data Cu (e.g., the most recent concentration data point received at step 110, which may be indicative of the material change in concentration) is actually a typographical or analytical error (e.g., determined at decision block 134). For example, the patient may be undergoing treatment for IBD. The patient's weight and CRP levels may be fine or improving but the concentration level of the infliximab in the patient's blood may appear to dramatically decrease. The system may determine the additional concentration data Cu (indicating the sudden decrease in infliximab in the patient's blood) was incorrectly entered into the system or there was an analytical error.

All five scenarios (briefly described above and described in further detail below) begins and ends with block 106, and includes at least blocks 104, 106, 108, 110 and/or 114, 112 and/or 116, 118, 119, and 120. As additional data is received (e.g., Cu at 110 and Pu at 114), the system determines whether there are changes in the data (e.g., changes in the concentration data at decision block 120 and whether the patient's physiological parameters are acceptable at decision block 122), or receives inputs from a medical professional that reflect whether there are such changes.

As discussed above, all of the scenarios begin at model block 106, which performs an iteration of the model, described below. An iteration of the model includes setting of the model parameters at block 104, which may include any suitable model parameters or coefficients of a pharmacokinetic model, a pharmacodynamic model, or a pharmacokinetic/pharmacodynamic model. The parameters may take into account any number of the inputs identified at block 102 and may further include additional information. For example, on the first execution of the system, the parameters may be set to account for all inputs (physiological parameters \vec{P}, target trough T, and concentration data \vec{C}). Additional inputs may include drug data indicative of a set of drugs, route of administration, or both. The first execution of the system may occur after the inputs are all received, after a set time period, or at any suitable time. In some aspects, on subsequent executions, the parameters may be updated to reflect physiological parameters \vec{P}, target trough T, and a subset of updated concentration data \vec{C}. One example of updating the inputs to include physiological parameters \vec{P}, target trough T, and a subset of updated concentration data \vec{C}, is described below in detail in relation to FIG. 5. In some aspects, on subsequent executions, the parameters may be updated to reflect physiological parameters \vec{P} and target trough T (and to exclude the updated concentration data \vec{C}). One example of updating the inputs to include the physiological parameters \vec{P} and target trough T while excluding the updated concentration data \vec{C} is described in detail in relation to FIG. 12. In some aspects, on subsequent executions, the parameters may be updated to consist of updated physiological parameters \vec{P}, target trough T, updated concentration data \vec{C}. In some implementations, block 104 corresponds to steps 304 and 312 of FIG. 3.

The model block 106 is a computational model that generates predictions of concentration time profiles of a drug in a patient. Model 106 may include a pharmacokinetic component indicative of a concentration time profile of the drug or class of drugs and a pharmacodynamic (e.g., response to treatment) component based on synthesis and degradation rates of a pharmacodynamic marker indicative of an individual response of the patient to the drug or class of drugs. Examples of models for such drugs are described in the '379 application. Model 106 may be selected from a set of computational models or may be a composite model incorporating aspects of multiple mathematical models, as described in the '545 application. Model 106 may be selected to best match the received physiological data. Model 106 may be a Bayesian model or any other suitable model.

In some implementations, the systems described herein may not be specific to a particular drug but instead apply to a class, or other subset or grouping of drugs (e.g., drugs that are expected to have a similar PK/PD, drugs known to be candidates of treating a particular condition, or other point of similarity). Models applicable to a class or other grouping of drugs may also be referred to as global models herein. Such global models may be predicated on one or more of several factors, including: 1) a common universal structural PK and/or PD model for all agents in a specific class, 2) similar effects of patient factors on the PK and/or PD parameters, and 3) similar indications. For example, the systems and methods described herein provide a pharmacokinetic model capable of being used for all biologics used in the treatment of inflammatory diseases. Such a model can be used to forecast dose regimens for fully human monoclonal antibodies (mAb), chimeric mAbs, fusion proteins, and mAb fragments (i.e., a range of drugs with differing pharmacokinetic properties but other similarities such as molecular weight and indication) using the same model. The model can be used in a broader patient population, including inflammatory bowel disease, rheumatoid arthritis, psoriatic arthritis, psoriasis, multiple sclerosis, and other such diseases that arise from immune dysregulation.

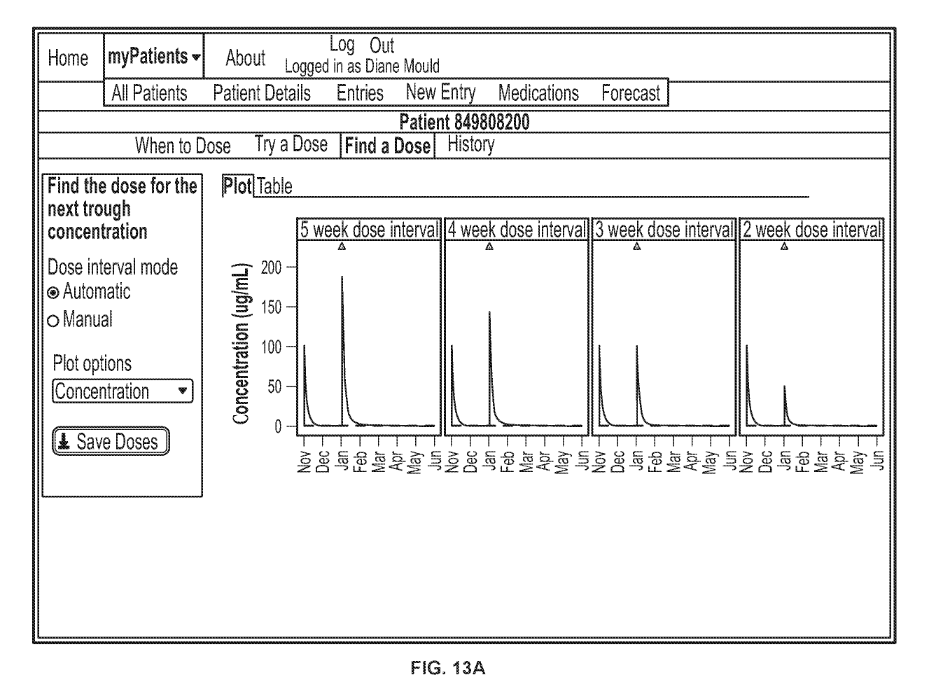

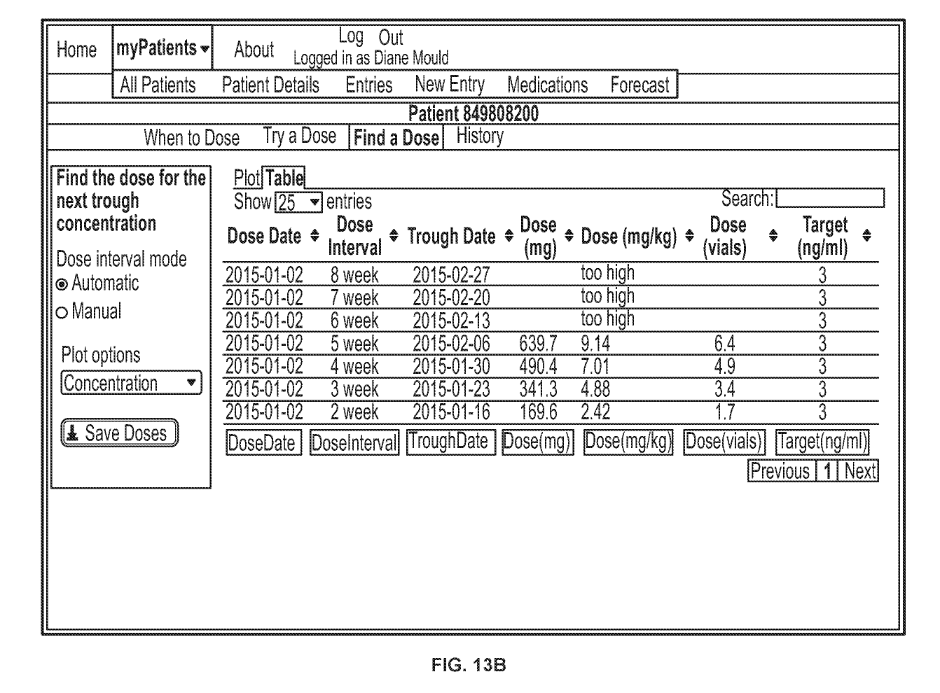

Model 106 provides a recommended dosing regimen to a user interface. The dosing regimen includes recommended times and doses to administer one or more pharmaceutical or drugs to the patient. One example of recommended times and doses is described in detail in relation to FIG. 13. The dosing regimen output by model 106 may correspond to the first pharmaceutical regimen determined in step 306 or the second pharmaceutical dosing recommendation of step 314 of FIG. 3. For example, the dosing regimen may be a first pharmaceutical dosing regimen comprising at least one dose amount of the drug and a recommended schedule for administering the at least one dose amount of the drug to the patient.

Upon viewing the recommended dosing regimen over the user interface, a medical professional may select to administer the recommending dosing regimen as recommended, or the medical professional may select to slightly alter the recommended dosing regimen, such as by changing one or more dates or times in the recommended schedule to accommodate the patient's or professional's schedule, and/or by changing the dosage amount (e.g., by rounding up to the nearest integer of vials, for example). A dosing regimen (e.g., either the recommended dosing regimen, or a modified version of the recommended dosing regimen) is administered by the medical professional, for example through oral medication; intravenous, intramuscular, intrathecal, or subcutaneous injection; insertion rectally or vaginally; infusion; topical, nasal, sublingual, or buccal application; inhalation or nebulization; the ocular route or the otic route; or any other suitable administration route. The dose amount may be a multiple of an available dosage unit for the drug. For example, the available dosage unit could be one pill or a suitable fraction of a pill that results when it is easily split, such as half a pill. In some implementations, the dose amount may be an integer multiple of the available dosage unit for the drug. For example, the available dosage unit could be a 10 mg injection or a capsule that cannot be split. For some routes of administration (e.g., IV and subcutaneous) any portion of the dose strength can be administered. The professional (or another user of the system) provides data indicative of the actual administered dosing regimen to the system at block 108. In an example, if the administered dosing regimen is the same as the recommended dosing regimen, the user may simply select a button on the user interface indicating the recommended dosing regimen was selected to be administered. Alternatively, if the administered dosing regimen is different from the recommended dosing regimen, the user provides data indicative of the administered dosing regimen to the system at block 108.

After a start of administration of the dosing regimen to the patient (which may be the same as or a modified version of the recommended dosing regimen provided by the model 106), the system receives additional data indicative of observed responses of the patient to the dosing regimen. In particular, the system may receive additional concentration data Cu at block 110, additional physiological data Pu at block 114, or both. The system receives additional concentration data Cu 110, which is concatenated at block 112 with the concentration data \vec{C}, such that \vec{C}=[\vec{C}; Cᵤ]. For example, the additional concentration data Cᵤ may be representative of the patient's response to the dosing regimen administered at block 108, and may be a single data point or multiple data points. Additionally or alternatively, the system receives additional physiological parameter data Pᵤ 114, which is concatenated at block 116 with the concentration data \vec{P}, such that \vec{P}=[\vec{P}; Pᵤ]. For example, the additional physiological parameter data Pᵤ may be representative of the patient's response to the dosing regimen administered at block 108, and may be a single data point, or multiple data points in a vector or a matrix form. Cᵤ 110 and Pᵤ 114 correspond to the additional concentration data and additional physiological data obtained from the patient in step 308 of FIG. 3. Concatenation 112 corresponds to step 316 of FIG. 3, where the concentration data is updated to include additional concentration data (e.g., additional concentration data Cu). From block 108, the system proceeds through blocks 110 and 112 to block 118, through blocks 114 and 116 to block 118, or through both sets of blocks (110, 112, 114, 116) to block 118. For example, the system might receive additional concentration data Cu without receiving additional physiological parameter data Pᵤ. In this example, the system would proceed through blocks 110 and 112 to update \vec{C} but would not necessarily need to update \vec{P} Alternatively, the system may receive additional physiological parameter data Pᵤ without receiving additional concentration data Cᵤ. In another example, the system may receive both Cᵤ and Pᵤ.

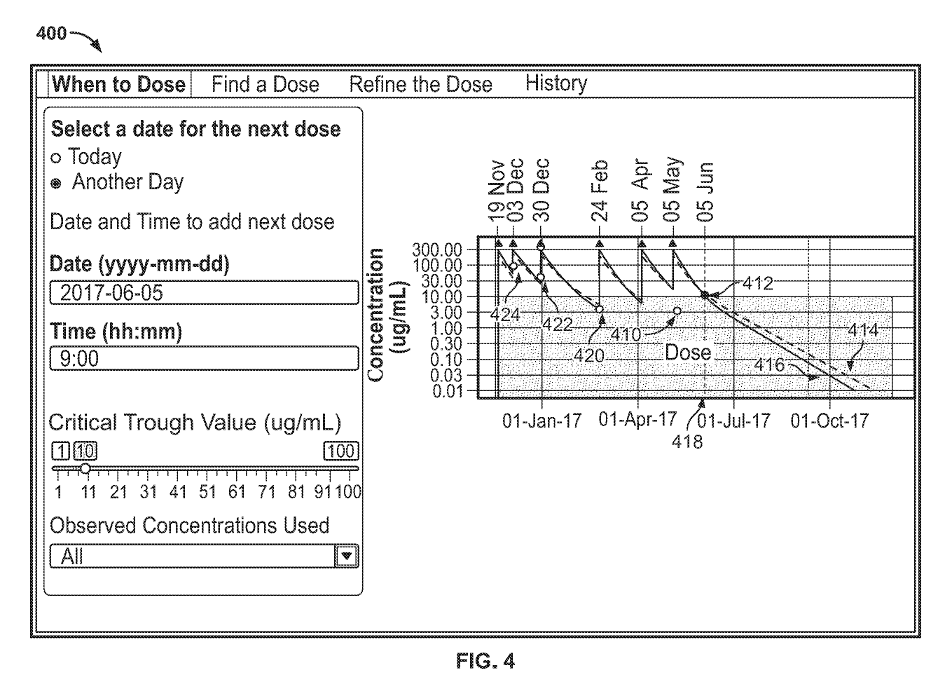

At block 118, the system displays indications of \vec{C} and/or \vec{P} on a user interface. The user interface may display the data points for concentration as measured at different times, one or more physiological parameters as determined at different times, or both. The user interface may include graphical displays, user input selections, dosing regimens, and any other suitable display. For example, the system may display a graph such as that shown in FIGS. 4-12 and described below. In some implementations, a user may toggle between different viewing windows on the user interface in order to view concentration and physiological parameter data.

At block 119, the system receives a first input indicative of whether there is a material change in \vec{C}. For example, the physician may determine a rate of change of the concentration data \vec{C}. The physician may compare the rate of change of the concentration data \vec{C} to a threshold rate of change value to determine the change in \vec{C} is sudden, and may then press a key on the user interface or press a key to indicate to the system that the change is material. In some implementations, the user may indicate that the change is material by entering a different mode of operation for the system. For example, the user may click a button to enter a “rapidly declining” mode for a patient.

The system also receives a second input indicative of the patient's health status. This may be determined based on whether the physiological parameter data \vec{P} is acceptable, or falls within a particular range or ranges. The physician may select a physiological parameter from the at least one physiological parameter indicated by physiological parameter data \vec{P}. For example, the physician may choose a physiological parameter in the user interface and view a graph of \vec{P} data corresponding to the chosen physiological parameter. For example, the physician may look at the patient's weight, and determine a patient is losing weight at a rate of 0.1 kg/day. The physician may compare the rate of change to a threshold rate of change value to determine whether the change in \vec{P} is “sudden.” As discussed above, the physician may use the quantifiable measurements in the physiological parameter data to assess the patient's health status. Additionally or alternatively, the physician may assess the patient's health status based on the patient's concentration data, qualitative assessments of the patient, or both.

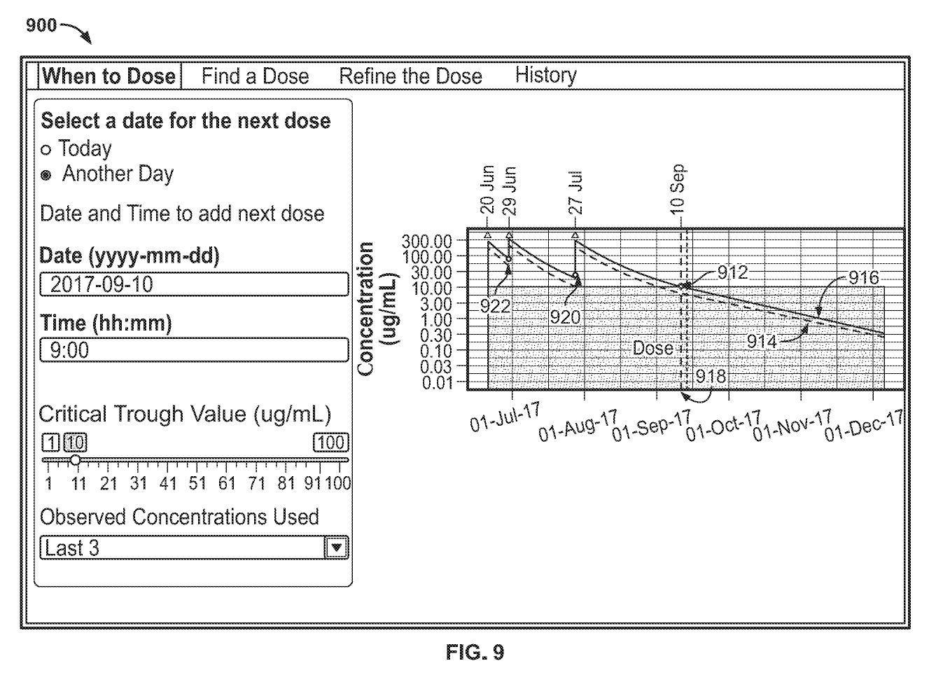

At decision block 120, the system determines whether the received first input (e.g., from a physician) indicates there has been a material change in the patient's concentration data. If not, the system proceeds to decision block 122, to determine whether the received second input indicates the patient's health status is acceptable. For example, after determining there has not been a material change in concentration, the physician may determine whether the patient's weight and CRP are in a healthy range. The physician may click a mouse or pressure a key on a user interface to indicate the physiological parameters are within acceptable ranges. The system may interpret this click or key input as indicating the physiological parameters are acceptable, Examples of determining whether the patient's physiological parameters are in a healthy range are described in detail in relation to FIGS. 9-11.

If the physiological parameters are acceptable as determined in decision block 122, the system proceeds to block 130 to set the inputs to the next model iteration as including all of the physiological parameter data, all of the concentration data, and the target response T. This concludes Scenario 1, as the system returns to block 106 to run another iteration of the model. The system automatically sets the parameters at block 104 to account for updates to the concentration data \vec{C} and/or the physiological parameter data \vec{P}.

Scenario 2 occurs when a patient's concentration data seems appropriate and has not suddenly changed (e.g., decision block 120 is “no”), but the patient's health status is not acceptable (e.g., decision block 122 is “no”). In this case, the system proceeds to block 126 to update the inputs to the next model iteration to include physiological parameter data \vec{P} and target trough T, while excluding all of the concentration data \vec{C}. In this scenario, a patient is not experiencing a material change in concentration but is still declining in health suggests that the target response may be inappropriate, such as being too low, as the patient's health status does not sufficiently improve. Accordingly, the concentration data \vec{C} is excluded from the model inputs for the next iteration to enable the system to determine a recommended dosing regimen based only on the clinical and laboratory data represented by the physiological parameter data. This may be desirable, for example, when the medical professional is aiming for an improper target trough concentration level in the patient. Removing the entirety of the concentration data \vec{C} from the model inputs allows the medical professional to react quickly to a failing patient (e.g., by making a dosing regimen determination based solely on laboratory data and/or clinical data) and gives the medical professional the opportunity to identify a new target response (e.g., a target trough concentration level) when the patient is more stable. This update to the system inputs and example situations leading to such an update are described in detail in relation to FIGS. 8 and 12 and step 310 of FIG. 3. From block 126, the system returns to block 106, where another iteration of the model is run and the model parameters are set. The system would then execute again, using the updated physiological parameters \vec{P} and target trough T, while not taking concentration data \vec{C} into account.

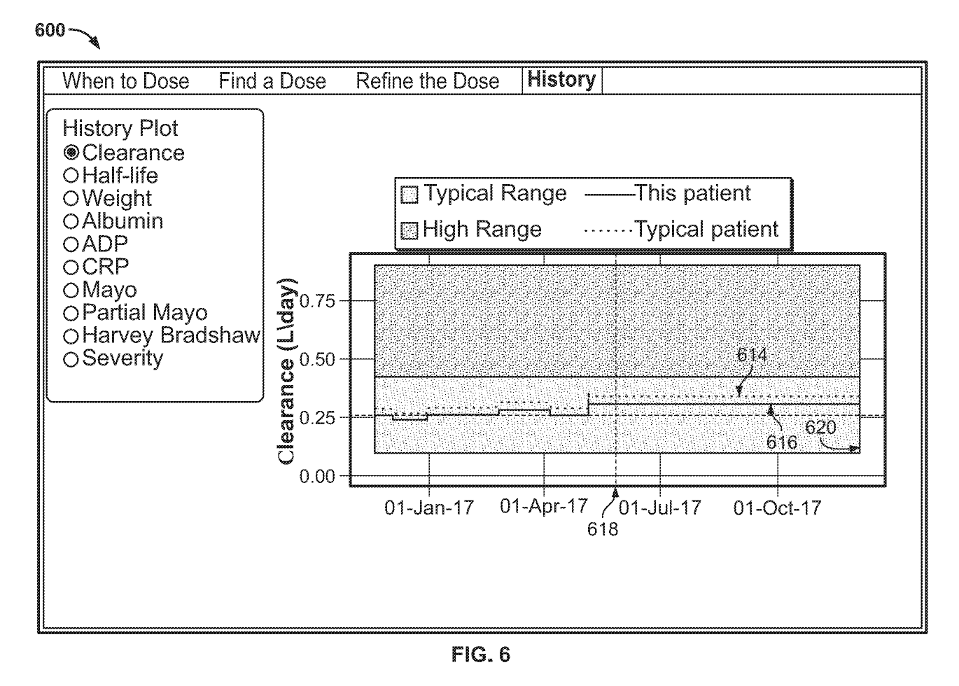

Scenario 3 occurs when the patient has taken a sudden turn for the worse. For example, the patient may be undergoing treatment for sepsis and develop kidney failure suddenly. In Scenario 3, a subset of the concentration data is excluded from the inputs to the model, leaving only the most recent concentration data to be included as input concentration data in the system's dosing regimen calculation. This input update ensures the system considers only the most recent concentration data to quickly react to the substantial turn for the worse in patient health, and further ensures the most recent concentration data point is not considered as an error. If there has been a material change in concentration (e.g., decision block 120 is “yes” and an example of which is described in detail in relation to FIGS. 4-5), the system proceeds to decision block 128 to determine whether the patient's health status is acceptable. For example, FIG. 6 shows acceptable elimination rates for the patient, which are within a typical elimination rate range. If the patient's health status is not acceptable (e.g., if the patient's physiological parameter data change rapidly or are out of the healthy range for a patient), the system continues in Scenario 3 (e.g., decision block 128 is “no”).

In this case, the system proceeds to block 132 to update the inputs to the system to include physiological parameters \vec{P}, target trough T, and recent concentration data of concentration data \vec{C}. The most recent concentration data is included (while the older concentration data is excluded), to ensure that the system reacts to the patient's current state (and most up-to-date information). This allows the system to react quickly to any change in the patient's health and does not neglect the most recent concentration data as an anomaly. As used herein, “recent” may include the last one, two, three, four, five, or any suitable number of data points in \vec{C}. Recent refers to the data points from the latest date and times available. This input update and the situations leading to such an update are described in detail in relation to FIG. 5 and step 320 of FIG. 3. From block 132, the system returns to block 106, where another iteration of the model is performed.

Alternatively, if the patient's health status is acceptable as determined in block 128 (e.g., decision block 128 is “yes”), the system proceeds to decision block 134 to determine whether additional concentration data Cᵤ is a typo or an anomaly. If Cᵤ is not a typo, the system enters Scenario 4. In this case, the patient experiences a material change in concentration but the patient's health status is acceptable, and the additional concentration data Cᵤ is not a typo or the result of an error, such as an assay error. For example, the patient may be undergoing treatment for IBD. The patient's weight and creatinine levels may be fine or improving but the concentration level of the infliximab in the patient's blood may dramatically decrease. From decision block 134, Scenario 4 proceeds to block 132, as described above in relation to Scenario 3, in which an older portion of the concentration data is excluded from the inputs to the next iteration of the model.

In some implementations, at decision block 134, the system determines whether the additional concentration data Cᵤ is consistent with the previously observed concentration data \vec{C}. If not, the system may prompt a user (such as a clinician or a clinician's assistant, for example) to check that the entered value for Cᵤ is accurate. While an existing system may simply ignore additional data that is not consistent with predictions (which causes the system's output to be skewed in favor of predictions instead of actual data), the present disclosure allows for a medical professional to confirm or fix the additional data, rather than simply ignoring or discarding it.

Alternatively, if the system determines that Cᵤ is a typo or an anomaly (which may be confirmed by a clinician or a user, as discussed above) at decision block 134, Scenario 5 proceeds to block 136 to remove Cᵤ from the concentration data \vec{C}. For example, the patient may be undergoing treatment for IBD. The patient's weight and CRP levels may be fine or improving but the concentration level of the infliximab in the patient's blood may appear to dramatically decrease. The system may determine the additional concentration data Cᵤ (indicating the sudden decrease in infliximab in the patient's blood) was incorrectly entered into the system. The system proceeds to block 136, to delete Cᵤ from the concentration data \vec{C}. The system completes Scenario 5 proceeding to block 138 to set the inputs to the next iteration of the model to reflect the physiological parameter data, the concentration data, and the target response T, before returning to block 106 to execute another iteration of the model.

In Scenario 5 or any situation in which the system determines Cᵤ is a typo or error, additional concentration data Cᵤ is entirely removed from the concentration data \vec{C}. On additional following iterations of the system (e.g., when the system restarts at block 106 and enters any of Scenarios 1 through 5), the particular value of Cᵤ will not be re-incorporated into the concentration data \vec{C}. For example, Cᵤ may be deleted from a memory storage device that stores \vec{C}. The removal of additional data Cᵤ differs from the exclusion of the older concentration data \vec{C} as discussed in relation to block 132 or the exclusion of all concentration data \vec{C} as discussed in relation to block 126. Unlike the removal of an error in block 136, the exclusion of all or part of concentration data \vec{C} may be temporary. For example, the older concentration data C may be excluded (e.g., in block 132) for three iterations of the model. On the fourth iteration, a physician may determine that the patient is now responding appropriately to treatment. In some implementations, the physician may input an indication the system should reincorporate the concentration data \vec{C} in its entirety, and no longer exclude the older concentration data. In some implementations, the system may automatically determine when the full set of the concentration data \vec{C} should again be considered.

Alternatively or in addition to block 119, the system may determine whether \vec{P} is acceptable. To check for changes in \vec{P}, the system compares the most recent additions to \vec{P} (e.g., Pᵤ) to the previous values in the data vectors \vec{P}. In some implementations, the system automatically determines this. In some implementations, the system is prompted to do this by an input from a clinician. In some implementations, the system may select a physiological parameter from the at least one physiological parameter indicated by physiological parameter data \vec{P}. In some implementation this parameter may be chosen by the physician or system user. The system may determine a rate of change of the selected physiological parameter of the patient and use this metric to determine whether \vec{P} has changed. For example, the system may look at the patient's weight, and determine a patient is losing weight at a rate of 0.1 kg/day. The system may compare the rate of change to a threshold rate of change value to determine whether the change in \vec{P} constitutes a sudden decline in health and whether the physiological parameters are acceptable. Thus at block 122 and 128, the second received input is determined by the system itself rather than received from a user.

Similarly, the system may determine a rate of change of the concentration data \vec{C}. To check for changes in \vec{C}, the system compares the most recent additions to \vec{C} (e.g., Cᵤ) to the previous values in the data vectors \vec{C} to determine whether the concentration has materially changed. In some implementations, the system automatically determines this. In some implementations, the system is prompted to do this by an input from a clinician. The system may determine a rate of change of the concentration of the drug in samples taken from the patient and use this metric to determine whether \vec{C} has materially changed. In some implementations, the system may calculate how much the most recent concentration data differs from the predicted concentration at that time to determine whether it has materially changed. Thus at block 120, the first received input is determined by the system itself rather than received from a user.

In some implementations, the system may determine whether may determine whether there \vec{P} is acceptable, while a physician inputs whether there is a material change in \vec{C}. In some implementations the system may determine whether there is a material change in \vec{C}, while the physician determines whether \vec{P} is acceptable. In some implementations, in contrast to the description of FIG. 1 above where both of these determinations are made by a system user, both determinations can be made by the system.

In some implementations, the system can “learn” from a physician's inputs (e.g., at block 119). For example, on the first several iterations of the system, the physician may manually need to input whether there is a material change in \vec{C} and whether \vec{P} is acceptable. On later iterations, the system may determine whether there is a material change in \vec{C} and whether \vec{P} is acceptable automatically, based at least partially on the previous inputs from the physician.

In some implementations, some of the decisions described above in relation to FIG. 1 are dependent on whether the patient is “early in treatment”. Example data regarding patients who are early in treatment are shown in FIGS. 7-8. In some implementations, a patient is considered to be early in treatment if an administered period of treatment of the patient with the drug is less than a threshold period of time. For example, a patient who has only been taking the drug for 1 hour, 6 hours, 12 hours, 1 day, 2 days, 3 days, 4 days, 5 days, 6 days, 1 week, 2 weeks, 3 weeks, 1 month, 2 months, 3 months or any suitable period of time might be considered early in treatment. In some implementations, a patient may be considered early in treatment if an administered period of treatment of the dosing regimen is less than a proportion of a total length of time of the dosing regimen. For example, if the total recommended length of time of the dosing regimen is N months, the patient may be considered to be early in treatment if the patient has been administered a dosing regimen for M months, where M is any number less than N. In an example, the proportion between M and N (e.g., M divided by N) may be 5%, 10%, 15%, 20%, 25%, 50%, 75%, or any other suitable proportion less than 100%. In an example, the patient may be considered to be early in treatment if the number of doses administered to the patient is less than a threshold proportion of a total number of doses to be administered. For example, N may correspond to a total number of doses scheduled to be administered to the patient, and M may correspond to the number of doses thus far administered to the patient.

If the patient is early in treatment, it may be desirable to exclude a larger portion of \vec{C} (e.g., increase the size of the subset excluded from the inputs) because the time span of concentration data \vec{C} is already short. When \vec{C} is already short, limiting the concentration data to a subset of \vec{C} to a broad recent set of \vec{C} may have no effect on the model's ability to provide improved dosing recommendations. For example, if there are three data points in concentration data \vec{C}, updating the concentration data \vec{C} to include only the three most recent data points will have no effect on the actual contents of \vec{C}. In another example, restricting c to several of the recent data points may not prevent the system from treating the last value as an outlier (and ignoring it). Instead, the system may limit \vec{C} to a single data point (e.g., the most recent data point collected). Removing all of the past \vec{C} data from the system inputs (apart from the most recent data point) guarantees that the system will not ignore the most recent data point. Instead, the system will rely on the patient's physiological state (as represented by the physiological parameter data F) and the single data point. For example, the patient may be undergoing treatment for sepsis (as described above) but has only received three doses. In this situation, it is appropriate to exclude the first two data points in the concentration data from the inputs to the system and to include only the most recent data point (and to update the parameters accordingly) to ensure the system quickly reacts to the worsening condition of the patient and does not treat the most recent (and suddenly changed) concentration data as an outlier.

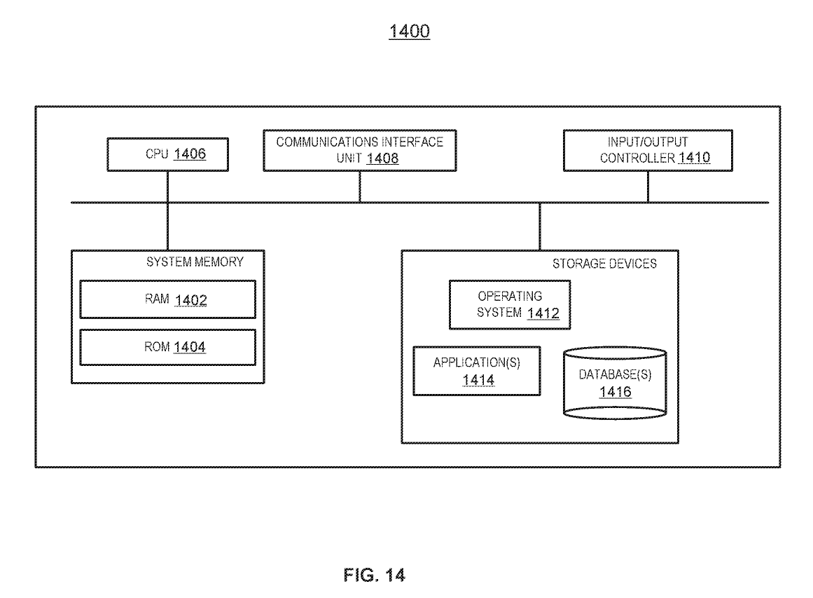

FIG. 2 shows a block diagram of a computerized system 200 for implementing the systems and methods disclosed herein. In particular, the system 200 uses drug-specific mathematical models and observed patient-specific responses to treatment to predict, propose, modify and evaluate suitable drug treatment plans for a specific patient. The system 200 includes a server 204, a clinical portal 214, a pharmacy portal 224, and an electronic database 206, all connected over a network 202. The server 204 includes a processor 205, the clinical portal 214 includes a processor 210 and a user interface 212, and the pharmacy portal 224 includes a processor 220 and a user interface 222. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that is currently being processed. An illustrative computing device 1400, which may be used to implement any of the processors and servers described herein, is described in detail below with reference to FIG. 14. As used herein, “user interface” includes, without limitation, any suitable combination of one or more input devices (e.g., keypads, touch screens, trackballs, voice recognition systems, etc.) and/or one or more output devices (e.g., visual displays, speakers, tactile displays, printing devices, etc.). As used herein, “portal” includes, without limitation, any suitable combination of one or more devices configured with hardware, firmware, and software to carry out one or more of the computerized techniques described herein. Examples of user devices that may implemental a portal include, without limitation, personal computers, laptops, and mobile devices (such as smartphones, blackberries, PDAs, tablet computers, etc.). For example, a portal may be implemented over a web browser or a mobile application installed on the user device. Only one server, one clinical portal 214, and one pharmacy portal 224 are shown in FIG. 2 to avoid complicating the drawing; the system 200 can support multiple servers and multiple clinical portals and pharmacy portals.

In FIG. 2, a patient 216 is examined by a medical professional 218, who has access to the clinical portal 214. The patient may be subject to a disease that has a known progression, and consults the medical professional 218. The medical professional 218 makes measurements from the patient 216 and records these measurements over the clinical portal 214. For example, the medical professional 218 may draw a sample of the blood of the patient 216, and may measure a concentration of a biomarker in the blood sample.

In general, the medical professional 218 may make any suitable measurement of the patient 216, including lab results such as concentration measurements from the patient's blood, urine, saliva, or any other liquid sampled from the patient. The measurement may correspond to observations made by the medical professional 218 of the patient 216, including any symptoms exhibited by the patient 216. For example, the medical professional 218 may perform an examination of the patient to gather or measure physiological parameters or to determine drug concentrations in the patient. This involves identifying patient characteristics (physiological parameters and concentrations) that are reflected as patient factor covariates within the mathematical model that will be used to predict the patient's response to a drug treatment plan. For example, if the model is constructed such that it describes a typical patient response as a function of weight and gender covariates, the patient's weight and gender characteristics would be identified. Any other characteristics may be identified that are shown to be predictive of response, and thus reflected as patient factor covariates, in the mathematical models. By way of example, such patient factor covariates may include markers of inflammation, an albumin measurement, an indicator of drug clearance, a measure of C-reactive protein (CRP), a measure of antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PASI) score and demographic information.

Based on the patient's measurement data, the medical professional 218 may make an assessment of the patient's disease status, and may identify a drug or class of drugs suitable for administering to the patient 216 to treat the patient 216. The clinical portal 214 may then transmit the patient's measurements, the patient's disease status (as determined by the medical professional 218), and, optionally, an identifier of the drug over the network 202 to the server 204, which uses the received data to select one or more appropriate computational models from the models database 206. The appropriate computational models are those that are determined to be capable of predicting the patient's response to the administration of the drug or class of drugs. The one or more selected computational models are used to determine a recommended set of planned dosages of the drug to administer to the patient, and the recommendation is transmitted back over the network 202 to the clinical portal 214 for viewing by the medical professional 218.

Alternatively, the medical professional 218 may not be capable of assessing the patient's disease status or identify a drug, and either or both of these steps may be performed by the server 204. In this case, the server 204 receives the patient's measurement data, and correlates the patient's measurement data with the data of other patients in the patient database 206a. The server 204 may then identify other patients who exhibited similar symptoms or data as the patient 216 and determine the disease states, drugs used, and outcomes for the other patients. Based on the data from the other patients, the server 204 may identify the most common disease states and/or drugs used that resulted in the most favorable outcomes, and provide these results to the clinical portal 214 for the medical professional 218 to consider.

As is shown in FIG. 2, the database 206 includes a set of four databases including a patient database 206a, a disease database 206b, a treatment plan database 206c, and a models database 206d. These databases store respective data regarding patients and their data, diseases, drugs, dosage schedules, and computational models. In particular, the patient database 206a stores measurements, including physiological parameter data and concentration data, taken by or symptoms observed by the medical professional 218. The disease database 206b stores data regarding various diseases and possible symptoms often exhibited by patients infected with a disease. The treatment plan database 206c stores data regarding possible treatment plans, including drugs and dosage schedules for a set of patients. The set of patients may include a population with different characteristics, such as weight, height, age, sex, and race, for example. The models database 206d stores data regarding a set of computational models that may be used to describe pharmacokinetic (PK), pharmacodynamic (PD), or both PK and PD changes to a body.

Any suitable computational model may be stored in the models database 206d, such as in the form of a compiled library module, for example. In particular, a suitable mathematical model is a mathematical function (or set of functions) that describes the relationship between a dosing regimen and the observed patient exposure and/or observed patient response (collectively “response”) for a specific drug. Accordingly, the mathematical model describes response profiles for a population of patients. Generally, development of a mathematical model involves developing a mathematical function or equation that defines a curve that best “fits” or describes the observed clinical data, as will be appreciated by those skilled in the art.

Typical models also describe the expected impact of specific patient characteristics on response, as well as quantify the amount of unexplained variability that cannot be accounted for solely by patient characteristics. In such models, patient characteristics are reflected as patient factor covariates within the mathematical model. Thus, the mathematical model is typically a mathematical function that describes underlying clinical data and the associated variability seen in the patient population. These mathematical functions include terms that describe the variation of an individual patient from the “average” or typical patient, allowing the model to describe or predict a variety of outcomes for a given dose and making the model not only a mathematical function, but also a statistical function, though the models and functions are referred to herein in a generic and non-limiting fashion as “mathematical” models and functions.

It will be appreciated that many suitable mathematical models already exist and are used for purposes such as drug product development. Examples of suitable mathematical models describing response profiles for a population of patients and accounting for patient factor covariates include PK models, PD models, hybrid PK/PD models, and exposure/response models. Such mathematical models are typically published or otherwise obtainable from drug manufacturers, the peer-reviewed literature, and the FDA or other regulatory agencies. Alternatively, suitable mathematical models may be prepared by original research. Moreover, as is described in the '545 Application, a Bayesian model averaging approach may be used to generate a composite model to predict patient response when multiple patient response models are available, though a single model may also be used.

In particular, the output of the model corresponds to a dosing regimen or schedule that achieves an optimal target level for a physiological parameter of the patient 216. The model provides the optimal target level as a recommendation specifically designed for the patient 216, and has verified that the optimal target level is expected to produce an effective and therapeutic response in the patient 216. In the example shown, the concentration data corresponds to a concentration of a drug in the patient's blood. In some implementations, the drug may not be known—for example, the drug may be any drug in a class of drugs. The physiological parameter data may correspond to any number of measurements from a patient. When the drug is infliximab, for example, it may be desirable to measure the drug concentration (and predict the drug concentration using the model) and other measurable units (that may be predicted by the model), such as C reactive protein, endoscopic disease severity, and fecal calprotectin. Each measurable (e.g., the drug concentration, C reactive protein, endoscopic disease severity, and fecal calprotectin) may involve one or more models, such as PK or PD models. The interaction between PK and PD models may be particularly important for a drug like infliximab, in which patients with more severe disease clear the drug faster (modeled by higher clearance from a PK model, as is explained in detail below). One goal of the drug infliximab may be to normalize C reactive protein levels, lower fecal calprotectin levels, and achieve endoscopic remission.

In one example, the medical professional 218 may assess the likelihood that the patient 216 will exhibit a therapeutic response to a particular drug and dosing regimen. In particular, this likelihood may be low if several dosing regimens of the same drug have been administered to the patient, but no measurable response from the patient is detected. In this case, the medical professional 218 may determine that it is unlikely that the patient will response to further adjustments to the dose, and other drugs may be considered. Moreover, a confidence interval may be assessed for the predicted model results and the predicted response of the body to the presence of the drug. As data is collected from the patient 216, the confidence interval gets narrower, and is indicative of a more trustworthy result and recommendation.

The systems and methods described herein may identify an individualized target level (e.g., target trough T of FIG. 1), and may provide individualized dosing recommendations based on the individualized target level.

Often, the medical professional 218 may be a member or employee of a medical center. The same patient 216 may meet with multiple members of the same medical center in various roles. In this case, the clinical portal 214 may be configured to operate on multiple user devices. The medical center may have its own records for the particular patient. In some implementations, the present disclosure provides an interface between the computational models described herein and a medical center's records. For example, any medical professional 218, such as a doctor or a nurse, may be required to enter authentication information (such as a username and password) or scan an employee badge over the user interface 212 to log into the system provided by the clinical portal 214. Once logged in, each medical professional 218 may have a corresponding set of patient records that the professional is allowed to access.

In some implementations, the patient 216 interacts with the clinical portal 214, which may have a patient-specific page or area for interaction with the patient 216. For example, the clinical portal 214 may be configured to monitor the patient's treatment schedule and send appointments and reminders to the patient 216. Moreover, one or more devices (such as smart mobile devices or sensors) may be used to monitor the patient's ongoing physiological data, and report the physiological data to the clinical portal 214 or directly to the server 204 over the network 202. The physiological data is then compared to expectations, and deviations from expectations are flagged. Monitoring the patient's data on a continual basis in this manner allows for possible early detection of deviations from expectations of the patient's response to a drug, and may indicate the need for modification of the dosing recommendation.

As described herein, the measurements from the patient 216 that are provided into the computational model may be determined from the medical professional 218, directly from devices monitoring the patient 216, or a combination of both. Because the computational model predicts a time progression of the disease and the drug, and their effects on the body, these measurements may be used to update the model parameters, so that the treatment plan (that is provided by the model) is refined and corrected to account for the patient's specific data.

In some implementations, it is desirable to separate a patient's personal information from the patient's measurement data that is needed to run the computational model. In particular, the patient's personal information may be protected health information (PHI), and access to a person's PHI should be limited to authorized users. One way to protect a patient's PHI is to assign each patient to an anonymized code when the patient is registered with the server 204. The code may be manually entered by the medical professional 218 over the clinical portal 214, or may be entered using an automated but secure process. The server 204 may be only capable of identifying each patient according to the anonymized code, and may not have access to the patient's PHI. In particular the clinical portal 214 and the server 204 may exchange data regarding the patient 216 without identifying the patient 216 or revealing the patient's PHI.

In some implementations, the clinical portal 214 is configured to communicate with the pharmacy portal 224 over the network 202. In particular, after a dosing regimen is selected to be administered to the patient 216, the medical professional 218 may provide an indication of the selected dosing regimen to the clinical portal 214 for transmitting the selected dosing regimen to the pharmacy portal 224. Upon receiving the dosing regimen, the pharmacy portal 224 may display the dosing regimen and an identifier of the medical professional 218 over the user interface 222, which interacts with the pharmacist 228 to fulfill the order.

As is shown in FIG. 2, the server 204 is a device (or set of devices) that is remote from the clinical portal 214. Depending on the computational power of the device that houses the clinical portal 214, the clinical portal 214 may simply be an interface that primarily transfers data between the medical professional 218 and the server 204. Alternatively, the clinical portal 214 may be configured to locally perform any or all of the steps described to be performed by the server 204, including but not limited to receiving patient symptom and measurement data, accessing any of the databases 206, running one or more computational models, and providing a recommendation for a dosage schedule based on the patient's specific symptom and measurement data. Moreover, while FIG. 2 depicts the patient database 206a, the disease database 206b, the treatment plan database 206c, and the models database 206d as being entities that are separate from the server 204, the clinical portal 214, or the pharmacy portal 224, one of ordinary skill in the art will understand that any or all of the databases 206 may be stored locally on any of the devices or portals described herein, without department from the scope of the present disclosure.

FIG. 3 shows a process 300 for an adaptive dosing system that determines a patient-specific pharmaceutical dosing regimen for a patient using a computerized pharmaceutical dosing regimen recommendation system that selectively removes existing data from the calculations under certain conditions, according to an illustrative implementation. The process 300 can be performed using the computerized system 200 of FIG. 2, or any other suitable computerized system. In step 302, inputs to the system are received. The inputs include (i) concentration data indicative of one or more concentration levels of a drug in one or more samples obtained from the patient, (ii) physiological data indicative of one or more measurements of at least one physiological parameter of the patient, and (iii) a target drug exposure level (e.g., a target concentration trough level). Further inputs to the system may also be received. For example, the process may be generic to a class of drugs, and additional inputs to the system may include drug information such as disease to be treated, class of drugs, route of administration, dose strength(s) available, preferred dosing amount (e.g., 100 mg vial), and/or whether the specific drug is fully human or not. In some implementations, historical data indicative of a response of the patient to a historical drug (e.g., a previously administered drug related to the currently administered drug) is received.

In step 304, parameters are set for a computational model that generates predictions of concentration time profiles of a drug in a patient. The computational model may be model 106 of FIG. 1, a PK/PD model, any of the models described above in relation to FIG. 2 (e.g., those stored in models database 206d), or any suitable model. The parameters are set based on the received inputs of step 302. In some implementations, the computational model is a Bayesian model. In some implementations, the computational model comprises a pharmacokinetic component indicative of a concentration time profile of the drug, and a pharmacodynamic component based on synthesis and degradation rates of a pharmacodynamic marker indicative of an individual response of the patient to the drug. In some implementations, process 300 comprises an additional, optional step where the computational model is selected from a set of computational models that best fits the received physiological data. In some implementations, the computational model accounts for the historical data indicative of a response of the patient to a historical drug in order to generate predictions of concentration time profiles of the drug in the patient. In some implementations, the historical drug may belong to the same class of drugs as the current drug. In some implementations, the historical drug may belong to a different class of drugs from the current drug.

In step 306, a first pharmaceutical dosing regimen for the patient is determined using the computational model and the set parameters. The first pharmaceutical dosing regimen comprises (i) at least one dose amount of the drug and (ii) a recommended schedule for administering the at least one dose amount of the drug to the patient. The recommended schedule includes a recommended time for administering a next dose of the drug to the patient, such that a predicted concentration time profile of the drug in the patient in response to the first pharmaceutical dosing regimen is at or above the target drug exposure level at the recommended time.

In step 308, additional concentration data and additional physiological data are obtained from the patient. The additional concentration data and additional physiological data may be obtained during or after administration of the first pharmaceutical dosing regimen to the patient. In some implementations, only additional concentration data is received at step 308, while in other implementations, only additional physiological data is received at step 308. In some implementations, process 300 comprises additional steps providing for the display of various system inputs and outputs. For example, the computational model may generate one or more predicted concentration time profiles of the drug in the patient's body in response to a pharmaceutical dosing regimen, and these one or more profiles may be displayed to a user. The same display may further include an indication of at least some of the patient's observed concentration data (e.g., on the user interface 222 in FIG. 2). In some implementations, an indication of the target drug exposure level is also displayed (e.g., on the user interface 222 in FIG. 2).

At decision block 322, the process diverges into two paths. The first path proceeds from step 322 to step 310 and then to step 312. The process 300 follows the first path when the patient's physiological parameters are not acceptable and there is not a material change in concentration level of the drug in the patient or the patient is early in treatment. FIG. 12 depicts an example of a predicted time concentration time profile (the first path), as described below, and as described above in relation to Scenario 2 of FIG. 1. In step 310, the inputs are updated to (i) exclude all of the concentration data from the inputs and (ii) include the additional physiological data in the inputs. Updating the inputs is based on the physiological data and the additional physiological data indicating a decline in health of the patient.

In some implementations, the decline in health is determined by selecting a physiological parameter from the at least one physiological parameter in physiological data and the additional physiological data of the patient; determining a rate of change of the selected physiological parameter of the patient; retrieving a threshold value from a database that correlates rates of change of physiological parameters with threshold values; and determining that the determined rate of change is greater than the retrieved threshold value. In some implementations determining the rate of change comprises identifying, from the physiological data and the additional physiological data, first data indicative of the selected physiological parameter of the patient at a first time and second data indicative of the selected physiological parameter of the patient at a second time; comparing the first data and the second data to determine an amount of change in the selected physiological parameter of the patient; determining a time interval between the first time and the second time; and determining, from the amount of change and the time interval, the rate of change of the selected physiological parameter of the patient. For example, the first time may be Aug. 1, 2017 at 9 am and the second time may be Aug. 11, 2017 at 9 am. On Aug. 1, 2017, the patient may weigh 100 lbs, but on Aug. 11, 2017 the patient may weigh 90 lbs. The rate of change in this patient's weight (a physiological parameter) is therefore 1 lb per day. The threshold may be 0.5 lbs per day. This 10 lbs decrease in 10 days is indicative of a decline in health in the patient, because the 1 lbs per day decreases surpasses the threshold.

The second path proceeds from decision block 322 to steps 316-320 and then to step 312. The process 300 follows the second path when there is a material change in concentration level of the drug in the patient, the patient's physiological parameters are not acceptable, and the patient is late in treatment. One example of a sudden and/or material change in concentration level of the drug in the patient is described in detail in relation to FIG. 4. FIG. 5 depicts an example of adjusting the inputs to the system, as described below, and as described above in relation to Scenarios 3 and 4 of FIG. 1. In step 316, the concentration data is updated to include both the concentration data of step 302 and the additional concentration data (e.g., Cᵤ) of step 308.

In step 318, the updated concentration data is divided into a subset (for inclusion in the model inputs for the next iteration of the model) and a remaining portion (for exclusion from the model inputs for the next iteration of the model). In this case, as discussed above in relation to Scenarios 3 and 4 of FIG. 1, it may be desirable to exclude an older set of concentration data from the next model iteration, while including the most recent set of concentration data, so that the next model iteration is sure to heavily account for the patient's recent concentration data in its determination of a recommended dosing regimen.

In step 320, the inputs are updated to (i) include the additional physiological data in the inputs, (ii) include the subset of the updated concentration data in the inputs (e.g., the most recent data points), and (iii) exclude the remaining portion of the updated concentration data from the inputs (e.g., the older data points). The update to the inputs at step 320 is based on the updated concentration data indicating a material change in the concentration level of the drug in the patient.

In step 312, the parameters for the computational model are updated, based on the updated inputs (e.g., similar to block 104 in FIG. 1), and in step 314, a second pharmaceutical dosing regimen for the patient is determined using the computational model and the updated parameters (e.g., similar to block 106 in FIG. 1, in which an iteration of the computational model is performed to determine a recommended dosing regimen for providing to the clinician or user).

FIGS. 4-12 described below are representative of various embodiments and examples of the above-described systems and methods. FIGS. 4-6 show results for an example patient, Patient A. Patient A took a sudden and substantial turn for the worse, exhibited by a material change in concentration data and physiological parameters that were not acceptable. Patient A's situation is described above in relation to Scenario 3 of FIG. 1 and steps 316-320 of FIG. 3. FIGS. 7-8 show results for an example patient, Patient B. Patient B took a sudden and substantial turn for the worse, exhibited by a material change in concentration data and was early in therapy. Patient B's situation is described above in relation to Scenario 5 of FIG. 1 and step 310 of FIG. 3. FIGS. 9-12 show results for an example patient, Patient C. Patient C's concentration data was matching expected values but the physiological parameters demonstrated a decline in health. Patient B's situation is described above in relation to Scenario 2 of FIG. 1 and step 310 of FIG. 3.

FIG. 4 shows an example display 400 of a user interface (e.g., user interface 212 of FIG. 2) on a clinical portal that provides a graph 402 of predicted concentration time profiles and recommended dosing regimens for a patient (Patient A), according to an illustrative implementation. The user may select a target trough value. For example, the user has selected a critical trough value of micrograms per milliliter (10 ug/mL), which is shown by the shaded portion of graph 402. The user may provide an input dosing regimen for testing by the model. For example, the user has set the next dosing date to be Jun. 5, 2017 at 9 am, because that is the date at which the predicted concentration time profile 416 intersects the critical trough value (shaded area). Alternatively, the system may provide the recommended next dosing date and dosing regimen.

The y-axis of graph 402 reflects concentration in ug/mL, while the x-axis of graph 402 reflects time. The concentration is indicative of any drug concentration of a class of drugs. For example, the class of drugs may be anti-inflammatory mAbs and the specific drug may be infliximab. Points 424, 422, 420, 410 represent concentrations as measured in the patient's blood (e.g., the concentration data described in step 302 of FIG. 3). In this case, the systems and methods of the present disclosure provide a predicted concentration profile curve 416 for Patient A based on the input dosing regimen, where the administered doses are indicated by the triangles at the top of graph 402. Curve 416 is thus the time concentration profile as predicted by the computerized recommendation system (e.g., the system described in relation to FIGS. 1-2), which takes into account past concentration measurements. For example, on Apr. 1, 2017, the system took into account not only physiological parameters data for Patient A, but also measured concentration data represented by points 424, 422, 420, 410. As additional measured concentration points are input to the system, the system will include these measurements when predicting patient response and recommending dosing regimens for Patient A. Line 418 indicates the next recommended date for dosing for Patient A. Point 412 represents the predicted concentration in the patient at the next date for dosing (Jun. 5, 2017) and intersects the critical trough value of 10 ug/mL.

Additionally, graph 402 displays curve 414, which is representative of a typical patient's concentration time profile, and is not based on the patient's individual concentration data measurements. The “typical patient” represented by curve 414 will have physiological parameters similar to that of Patient A in some situations. The system may also receive physiological parameter data for the patient. One example of physiological parameter data for the patient is described in detail in relation to FIG. 6. The physiological parameter data may be accounted for in the predicted concentration time profiles 414, 416.

As can be seen in graph 402, Patient A's condition changed markedly and to such an extent that the patient factors included in the model could not account for the change. The discrepancy between measured concentration 410 and predicted concentration 416 at the same point in time indicates Patient A has taken a sudden and substantial “turn for the worse” and Patient A's disease is progressing faster than would be expected, given the prior concentration data (points 424, 422, 420) as well as previously measured physiological parameters (not shown). As such, the model essentially treated the last observation as an outlier, which is why the point 410 is so far below the predicted concentration shown by curve 416 at the same point in time. Because the model treated point 410 as an outlier and did not account for it when providing a dosing recommendation, this leads to an overly optimistic assessment of the next date to dose Patient A.

The model is more likely to treat point 410 as an outlier (and ignore it) because the data from the earlier doses (points 424, 422, 420) showed good agreement with the model's predicted concentration (curve 416). Because the last observation 410 is considered an outlier and is essentially ignored, the next recommended dose at line 418 and predicted concentration 412 will also likely not align with Patient A's next measures concentration. However, the time span of concentration data used to forecast a patient's dose can be limited (as will be described below in relation to FIG. 5), improving the agreement between the observed data and the model prediction, and providing a better estimate of the next time to administer drug without sacrificing the precision of the individual patient parameters.

FIG. 5 shows a display 500 of a plot of drug concentration over time after the dosing regimen shown in FIG. 4 (and described above) has been modified. Display 500 may be displayed on a user interface (e.g., user interface 212 of FIG. 2). Again, the user selected critical trough value is micrograms per milliliter (10 ug/mL), which is shown by the shaded portion of graph 502. The user may provide an input dosing regimen for testing by the model. For example, the user has set the next dosing date to be May 27, 2017 at 9 am, because that is the date at which the predicted concentration time profile 516 intersects the critical trough value (shaded area). Alternatively, the system may provide the recommended next dosing date and dosing regimen. The y-axis of graph 502 reflects concentration in ug/mL, while the x-axis of graph 502 reflects time. Points 524, 522, 520, 510 represent concentrations as measured in the patient's blood. For example, the concentration may be indicative of a concentration of a specific drug (e.g., Infliximab) in a class of drugs present in the patient's blood stream. In this case, the systems and methods of the present disclosure provide a predicted concentration profile curve 516 for Patient A based on the input dosing regimen, where the administered doses are indicated by the triangles at the top of graph 502. Curve 516 is thus the time concentration profile as predicted by the computerized recommendation system (e.g., the system described in relation to FIGS. 1-2), which takes into account past concentration measurements. However, in this case, unlike in FIG. 4, the dosing recommendations system only takes into account the two most recent concentration data points 420 and 410. Concentration data points 422 and 424 are not included as parameters into the model. In some implementations, the subset of concentration data may comprise the three most recent concentration data measurements, the two most recent, or any suitable number of recent measurements. In some implementations, the system may constantly limit the number of data points comprising the concentration data to the three most recent concentration data measurements.

As additional measured concentration points are input to the system, the system will include these new measurements when predicting patient response and recommending dosing regimens for Patient A, while excluding older concentration data points. For example, the system may only account for the last one, two, three or any suitable number of concentration measurements. With a limited number of observations used to base the Bayesian update, the last observation becomes a meaningful percentage of the data and is therefore given more weight during the Bayesian update.

Line 518 indicates the next recommended date for dosing for Patient A. Point 512 represents the predicted concentration in the patient at the next date for dosing (May 27, 2017) and intersects the critical trough value of 10 ug/mL.

Additionally, graph 502 displays curve 514, which is representative of a typical patient's concentration time profile, and is not based on the patient's individual measurements (concentration data or physiological parameter data).

As can be seen in graph 502, unlike in FIG. 4, once the concentration data was no longer included as a model parameter, the model no longer treated the most recent concentration data point 510 as an outlier. In fact, the most recent measurements were given more “weight” in the model by decreasing the total number of measurements accounted for. The point 510 thus more closely matched predicted concentration shown by curve 516 at the same point in time. The system has therefore accounted for Patient A's sudden and substantial “turn for the worse.”

FIG. 6 shows display 600 of a plot of Patient A's drug clearance over time. Display 600 may be displayed on a user interface (e.g., user interface 212 of FIG. 2). The y-axis of plot 602 is indicative of clearance in liters per day (L/day), while the x-axis is indicative of time. The clearance is applicable to any drug of a class of drugs. For example, the class of drugs may be anti-inflammatory mAbs and the specific drug may be infliximab. Patient A's estimated drug clearance over time shows an upward trend over time. Ideally, a patient's drug clearance should be slowing down or at least be stable over time. The upward trend in plot 602 indicates that the patient is not in good health and that the dosing regimen may need adjusted (as described above in relation to FIG. 5).

FIG. 7 shows a display 700 of a graph 702 of a predicted concentration time profile for a specific patient, Patient B, based on a subset of concentration data and physiological parameter data, according to an illustrative implementation. Display 700 may be displayed on a user interface (e.g., user interface 212 of FIG. 2). The y-axis of graph 702 reflects concentration in ug/mL, while the x-axis of graph 702 reflects time. The concentration is indicative of any drug concentration of a class of drugs. For example, the class of drugs may be anti-inflammatory mAbs and the specific drug may be infliximab. Points 722, 720 represent concentrations as measured in the patient's blood. The systems and methods of the present disclosure provide a predicted concentration profile curve 716 for Patient B based on the dosing regimen, where the administered doses are indicated by the triangles at the top of graph 702. The dosing regimen and predicted concentration time profile in this case are based off a subset of the total concentration data for patient B. Patient B is early in therapy and has only received three drug doses. Early in therapy a patient (e.g., Patient B) can worsen rapidly. However, when a patient is so early in therapy, truncating the concentration data, as described above in relation to FIG. 5, is unlikely to improve the model's ability to provide reasonable dose recommendations and accurately predict the patient's concentration time profile. The model will likely continue to treat the last concentration data measurement (such as the additional concentration data Cᵤ described above in relation to FIG. 1) as an outlier owing to the substantial change in concentration that is not accounted for by the previous concentration data or physiological data.

Curve 716 is thus the time concentration profile as predicted by the computerized recommendation system (e.g., the system described in relation to FIGS. 1-2), which takes into account a subset of past concentration measurements. Based on the subset of concentration measurements and the physiological data, the model has predicted that the concentration will reach the critical trough value on Oct. 7, 2017 at point 712. Based on this prediction, a medical professional should administer the dose ahead of or on Oct. 7, 2017. However, this prediction does not account for the observed concentration data at point 720, as evidenced by the difference in drug concentration between point 720 and curve 716 at the same time.

Additionally, graph 702 displays curve 714, which is representative of a typical patient's concentration time profile, and is not based on the patient's individual concentration data measurements.

FIG. 8 shows a graph 800 of a predicted concentration time profile for a specific patient, Patient B, based on physiological parameter data, according to an illustrative implementation. Graph 800 may be displayed on a user interface (e.g., user interface 212 of FIG. 2). The y-axis of graph 802 reflects drug concentration in ug/mL, while the x-axis of graph 802 reflects time. Points 822, 820 represent drug concentrations as measured in the patient's blood, and are the same as points 722, 720 of FIG. 7. The systems and methods of the present disclosure provide a predicted concentration profile curve 816 for Patient B based on the dosing regimen, where the administered doses are indicated by the triangles at the top of graph 800.

Curve 816 matches curve 714 of FIG. 7. The predicted concentration profile curve 816 is based on the physiological parameter data of Patient B and, unlike predicted concentration profile curve 716 of FIG. 7, it is not based on the concentration data of Patient B. By removing the concentration from the parameters considered by the model, the model is able to more accurately account for Patient B's current status in this situation, where Patient B is early in therapy and their concentration has suddenly changed from the expected value. This is shown by the close match between measured concentration data 820 on Sep. 1, 2017 and predicted concentration time profile curve 816 on this same date.

Based on the physiological data, the model has predicted that the concentration will reach the critical trough value on Sep. 15, 2017 at point 812. Based on this prediction, a medical professional should administer the dose ahead of or on Sep. 15, 2017, rather than ahead of or on Oct. 7, 2017 as was predicted in FIG. 7 using the physiological data and a subset of the concentration data.

FIG. 9 shows an example display 900 of a user interface (e.g., user interface 212 of FIG. 2) on a clinical portal that provides a graph 902 of a predicted concentration time profile for a patient, Patient C, according to an illustrative implementation. As described above, the user may select a target trough value. For example, the user has selected a critical trough value of micrograms per milliliter (10 ug/mL), which is shown by the shaded portion of graph 902. The user may provide an input dosing regimen for testing by the model. For example, the user has set the next dosing date to be Sep. 10, 2017 at 9 am, because that is the date at which the predicted concentration time profile 916 intersects the critical trough value (shaded area) as marked by point 912.

The y-axis of graph 902 reflects concentration in ug/mL, while the x-axis of graph 902 reflects time. Points 922, 920, 910 represent concentrations as measured in the patient's blood. For example, points 922, 920, 910 may represent infliximab concentrations. In this case, the systems and methods of the present disclosure provide the predicted concentration profile curve 916 for Patient C, where the administered doses are indicated by the triangles at the top of graph 902. Curve 916 is thus the time concentration profile as predicted by the computerized recommendation system (e.g., the system described in relation to FIGS. 1-2), which takes into account past concentration measurements and physiological parameter measurements.

Additionally, graph 902 displays curve 914, which is representative of a typical patient's concentration time profile, and is not based on the patient's individual concentration data measurements. The “typical patient” represented by curve 914 will have physiological parameters similar to that of Patient C in some implementations.

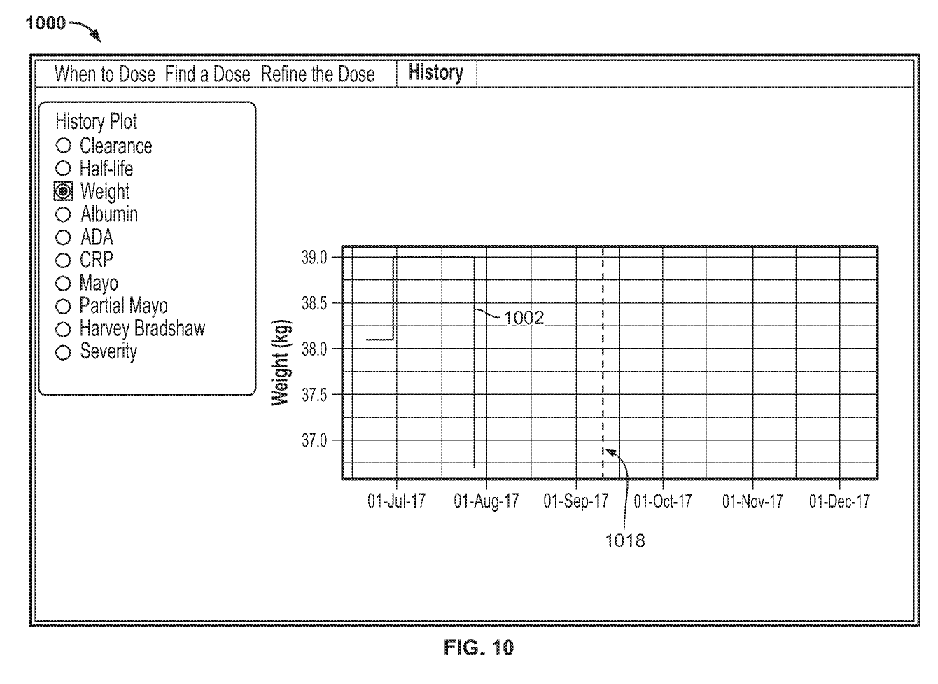

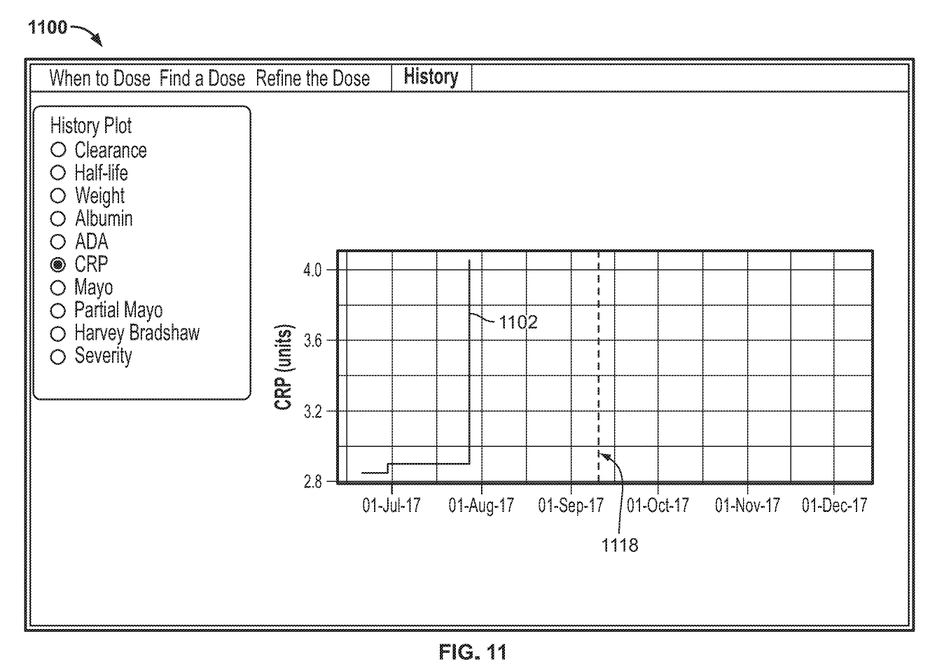

Points 922 and 920 closely match the predicted concentration time profile curve 916 at their respective measurement times. The measured concentration data points 922 and 920 are also well above the shaded critical trough value of 10 ug/mL. Thus the model is in good agreement with the observed concentration measurements and Patient C should be in good health. However, as will be described below in relation to FIGS. 10-11, Patient C health was not good during these measurements, and Patient C was getting worse during this treatment.

FIG. 10 shows a graph 1000 of Patient C's weight over time. Graph 1000 may be displayed on a user interface (e.g., user interface 212 of FIG. 2). The y-axis of graph 1000 shows Patient C's weight in kilograms (kg), while the x-axis shows time. The time frame depicted in graph 1000 is the same as that depicted in graph 902 of FIG. 9. Patient C's weight decreases overall during administration of the drug, which may be an indication of Patient C's declining health.

FIG. 11 shows a graph 1100 of patient C-reactive protein over time. Graph 1100 may be displayed on a user interface (e.g., user interface 212 of FIG. 2). The y-axis of graph 1100 shows Patient C's CRP in units, while the x-axis shows time. The time frame depicted in graph 1100 is the same as the depicted in graphs 902 and 1000 of FIGS. 9 and 10 respectively. CRP is a marker of inflammation. Patient C's CRP increases over time, which may be an indication of Patient C's declining health.

In some implementations, to determine whether a patient is experiencing a decline in health, the system may select a physiological parameter from the at least one physiological parameter in physiological data and the additional physiological data of the patient. The system may determine a rate of change of the selected physiological parameter of the patient. In some implementations, to determine the rate of change, the system may identify first data indicative of the selected physiological parameter of the patient at a first time and second data indicative of the selected physiological parameter of the patient at a second time. The system may compare the first data and the second data to determine an amount of change in the selected physiological parameter of the patient, determine a time interval between the first time and the second time, and determining, from the amount of change and the time interval, the rate of change of the selected physiological parameter of the patient. The rate of change may be compared to a threshold value for that physiological parameter. If the rate of change is greater than the threshold, the system may determine the patient is experiencing a decline in health. For example, the selected physiological parameter for Patient C may be weight, as displayed in FIG. 10. The first data may be Patient C's weight measured at 38.1 kg at first time Jun. 20, 2017. The second data may be Patient C's weight measured at 36.7 kg at second time Jul. 25, 2017. The rate of change for Patient C would be 1.4 kg in 35 days, or 0.04 kg per day. The threshold for weight may be 0.02 kg per day. Such a threshold may indicate that a weight loss greater than 0.02 kg per day is suggestive of a decline in health of the patient. Because the rate of change exceeds the threshold in this example, the system would determine Patient C is determining a decline in health.

Patient C's apparent decline in health indicated by the decreasing weight shown in FIG. 10 and increasing CRP shown in FIG. 11, may indicate the target trough concentration was chosen incorrectly. In a clinical situation, a medical professional may not have time to determine a new appropriate target trough concentration. The concentration data may be temporarily excluded from any weighting in the system to allow the dosing regimen to be adjusted based on the physiological parameter data, as shown below in FIG. 12. In some implementations, the concentration data collected for the patient (e.g., Patient C) can be used to later determine a better target trough. Removing the concentration data from the inputs to the system allows the medical professional to react quickly to an apparently failing patient, and gives the medical professional time to identify a new target at a later date when the patient begins to respond more positively to a dosing regimen.

FIG. 12 shows a graph 1200 of a predicted concentration time profile for Patient C, based on physiological parameter data, according to an illustrative implementation. Graph 1200 may be displayed on a user interface (e.g., user interface 212 of FIG. 2). Removing the concentration data from the system inputs results in a shorter dose interval and an earlier time to administer the next dose than was predicted for Patient C in FIG. 9 (where concentration data was included as an input to the system). In a setting where a patient (e.g., Patient C) has concentration data matching expected results but is still failing, it is appropriate to adjust the dosing regimen based on the patient's physiological parameter data.

FIG. 13A depicts an example display screen (e.g., user interface 212 of FIG. 2) that displays predicted concentration time profiles for four different dosing regimens. In particular, each dosing regimen has a corresponding dose interval (5 weeks, 4 weeks, 3 weeks, and 2 weeks), where the dose amount decreases as dose interval decreases (as is indicated by the height of the second peak in each predicted concentration time profile). In this case, the user has selected to plot IFX concentration versus time, and to allow the computational models to run to identify recommended dosing regimens to maintain IFX concentrations above the critical trough value.

In FIG. 13B, the user has selected to display the results from the plot shown in FIG. 13A in a table form (e.g., on user interface 212 of FIG. 2). In particular, the example display screen in FIG. 13B lists the last dose date (Jan. 2, 2015), various dosing intervals, a trough date (corresponding to the first date after the dose date that the predicted concentration time profile falls to or at the critical trough value), the suggested dose (in mg), the normalized suggested dose (in mg/kg), the number of vials used for each dose, and the target concentration (in ng/ml). As is shown in FIG. 13B, a set of proposed dosing schedules is shown, where the dosing schedules have different dose intervals ranging from two to eight weeks. While some of the dosing schedules with longer dose intervals (six to eight weeks) are not recommended, four dosing schedules (with dose intervals of two to five weeks) are proposed with doses that increase as dose interval increases. In particular, when interacting with the display screen of FIG. 13B, the medical professional 218 may select a dosing regimen based on a specific goal. For example, the longer dose interval (e.g., five weeks) may be selected if it is desirable to administer doses to the patient 216 infrequently. Alternatively, since patients are often charged the price of a full vial, even when a partial vial is used, it may be desirable to use as much of the vials as possible. In this case, the four-week dosing regimen may be selected, since 4.9 vials are used for each dose, and leads to little wastage of the drug (e.g., only 0.1 vials per dosage). Alternatively, a shorter dose interval (e.g., two weeks) may be selected it if is desirable to administer doses to the patient 216 more frequently, or to charge the patient 216 for only two vials at a time.

FIG. 14 is a block diagram of a computing device, such as any of the components of the systems of FIG. 2, for performing any of the processes described herein. Each of the components of these systems may be implemented on one or more computing devices 1400. In certain aspects, a plurality of the components of these systems may be included within one computing device 1400. In certain implementations, a component and a storage device may be implemented across several computing devices 1400.

The computing device 1400 includes at least one communications interface unit, an input/output controller 1410, system memory, and one or more data storage devices. The system memory includes at least one random access memory (RAM 1402) and at least one read-only memory (ROM 1404). All of these elements are in communication with a central processing unit (CPU 1406) to facilitate the operation of the computing device 1400. The computing device 1400 may be configured in many different ways. For example, the computing device 1400 may be a conventional standalone computer or alternatively, the functions of computing device 1400 may be distributed across multiple computer systems and architectures. In FIG. 1400, the computing device 1400 is linked, via network or local network, to other servers or systems.

The computing device 1400 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 1408 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 1406 includes a processor, such as one or more conventional microprocessors and one or more supplementary co-processors such as math co-processors for offloading workload from the CPU 1406. The CPU 1406 is in communication with the communications interface unit 1408 and the input/output controller 1410, through which the CPU 1406 communicates with other devices such as other servers, user terminals, or devices. The communications interface unit 1408 and the input/output controller 1410 may include multiple communication channels for simultaneous communication with, for example, other processors, servers or client terminals.

The CPU 1406 is also in communication with the data storage device. The data storage device may include an appropriate combination of magnetic, optical or semiconductor memory, and may include, for example, RAM 1402, ROM 1404, flash drive, an optical disc such as a compact disc or a hard disk or drive. The CPU 1406 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 1406 may be connected to the data storage device via the communications interface unit 1408. The CPU 1406 may be configured to perform one or more particular processing functions.

The data storage device may store, for example, (i) an operating system 1412 for the computing device 1400; (ii) one or more applications 1414 (e.g., computer program code or a computer program product) adapted to direct the CPU 1406 in accordance with the systems and methods described here, and particularly in accordance with the processes described in detail with regard to the CPU 1406; or (iii) database(s) 1416 adapted to store information that may be utilized to store information required by the program.

The operating system 1412 and applications 1414 may be stored, for example, in a compressed, an uncompiled and an encrypted format, and may include computer program code. The instructions of the program may be read into a main memory of the processor from a computer-readable medium other than the data storage device, such as from the ROM 1404 or from the RAM 1402. While execution of sequences of instructions in the program causes the CPU 1406 to perform the process steps described herein, hard-wired circuitry may be used in place of, or in combination with, software instructions for implementation of the processes of the present invention. Thus, the systems and methods described are not limited to any specific combination of hardware and software.

Suitable computer program code may be provided for performing one or more functions described herein. The program also may include program elements such as an operating system 1412, a database management system and “device drivers” that allow the processor to interface with computer peripheral devices (e.g., a video display, a keyboard, a computer mouse, etc.) via the input/output controller 1410.

The term “computer-readable medium” as used herein refers to any non-transitory medium that provides or participates in providing instructions to the processor of the computing device 1400 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 1406 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 1400 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

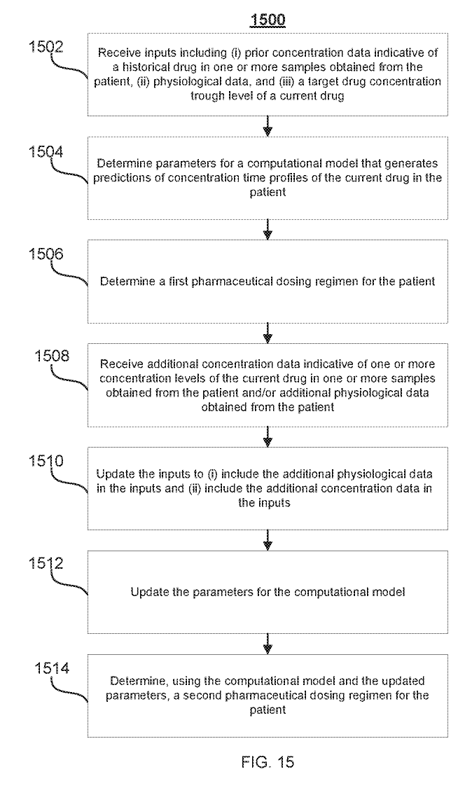

FIG. 15 shows a process 1500 for using data from a patient's response to a prior treatment, to inform a recommended dosing regimen for a different treatment, according to an illustrative implementation. The process 1500 can be performed using the computerized system 200 of FIG. 2, or any other suitable computerized system. In step 1502, inputs to the system are received. The inputs include prior concentration data indicative of one or more prior concentration levels of a prior drug in one or more samples obtained from the patient, physiological data indicative of one or more measurements of at least one physiological parameter of the patient, and a target drug exposure level of a current drug. A patient may have failed therapy on the prior drug. Medical information (e.g., concentration data) indicating the patient's response to the prior drug may be useful in helping to determine how a patient will react to a current drug. In some implementations, the prior and current drug may belong to the same class or may have similar effects. For example, the prior and current drugs may be different monoclonal antibodies. If a patient's data indicates that the patient has a high elimination rate for the prior drug (e.g., the patient's body eliminates the prior drug from the patient's system at a higher rate than average or than is expected), then the patient is likely to have a similarly high elimination rate for the current drug. By retaining information from the prior drug, the systems and methods described herein may quickly determine an individualized dosing regimen for the current drug, thereby reducing the amount of time a patient is untreated if switching between drugs.

In step 1504, parameters are set for a computational model that generates predictions of concentration time profiles of the current drug in a patient. The computational model may be model 106 of FIG. 1, a PK/PD model, any of the models described above in relation to FIG. 2 (e.g., those stored in models database 206d), or any suitable model. The parameters are set based on the received inputs of step 1502. In some implementations, the computational model is a Bayesian model. In some implementations, the computational model comprises a pharmacokinetic component indicative of a concentration time profile of the drug, and a pharmacodynamic component based on synthesis and degradation rates of a pharmacodynamic marker indicative of an individual response of the patient to the drug. In some implementations, process 1500 comprises an additional, optional step where the computational model is selected from a set of computational models that best fits the received physiological data.

In step 1506, a first pharmaceutical dosing regimen for the patient is determined using the computational model and the set parameters. The first pharmaceutical dosing regimen comprises (i) at least one dose amount of the drug and (ii) a recommended schedule for administering the at least one dose amount of the drug to the patient. The recommended schedule includes a recommended time for administering a next dose of the drug to the patient, such that a predicted concentration time profile of the drug in the patient in response to the first pharmaceutical dosing regimen is at or above the target drug concentration trough level at the recommended time.

In step 1508, additional concentration data corresponding to the current drug and/or additional physiological data are obtained from the patient. The additional concentration data and additional physiological data may result from administration of the first pharmaceutical dosing regimen to the patient. In some implementations, process 1500 comprises additional steps providing for the display of various system inputs and outputs. In some implementations, process 1500 provides the predicted concentration time profile of the drug in the patient in response to the first pharmaceutical dosing regimen, as generated by the computational model, and an indication of at least some of the concentration data and the additional concentration data for display (e.g., through user interface 222 in FIG. 2). In some implementations, an indication of the target drug concentration trough level is provided for display (e.g., through user interface 222 in FIG. 2).

In step 1510, the concentration data is updated to include both the concentration data of step 1502 and the additional concentration data of step 1508. In some implementations, at step 1510, the original concentration data from step 1502 is excluded, and the additional concentration data becomes the concentration data.

In step 1512, the parameters for the computational model are updated, based on the updated inputs, and in step 1514, an iteration of the model is performed to determine a second pharmaceutical dosing regimen for the patient, based on the updated parameters.

It is to be understood that while various illustrative implementations have been described, the forgoing description is merely illustrative and does not limit the scope of the invention. While several examples have been provided in the present disclosure, it should be understood that the disclosed systems, components and methods of manufacture may be embodied in many other specific forms without departing from the scope of the present disclosure.

The examples disclosed can be implemented in combinations or sub-combinations with one or more other features described herein. A variety of apparatus, systems and methods may be implemented based on the disclosure and still fall within the scope of the invention. Also, the various features described or illustrated above may be combined or integrated in other systems or certain features may be omitted, or not implemented.

While various implementations of the present disclosure have been shown and described herein, it will be obvious to those skilled in the art that such implementations are provided by way of example only. Numerous variations, changes, and substitutions will now occur to those skilled in the art without departing from the disclosure. It should be understood that various alternatives to the implementations of the disclosure described herein may be employed in practicing the disclosure.

All references cited herein are incorporated by reference in their entirety and made part of this application.

1. A method of determining a patient-specific pharmaceutical dosing regimen for a patient, using a computerized pharmaceutical dosing regimen recommendation system, the method comprising:
(a) receiving inputs including (i) concentration data indicative of one or more concentration levels of a drug in one or more samples obtained from the patient, wherein the drug is one of a set of drugs expected to exhibit similar pharmacokinetic (PK) behavior, similar pharmacodynamic (PD) behavior, or both, (ii) physiological data indicative of one or more measurements of at least one physiological parameter of the patient, and (iii) a target drug exposure level;
(b) determining, based on the received inputs, parameters for a computational model that generates predictions of concentration time profiles of the drug in the patient-, wherein the computational model is representative of responses by a plurality of patients to a plurality of drugs in the set of drugs, wherein each response of the responses is indicative of a patient response to at least one drug in the set of drugs, and wherein the computational model is not specific to a particular drug;
(c) determining, using the computational model and based on the determined parameters, a first pharmaceutical dosing regimen for the patient, wherein the first pharmaceutical dosing regimen comprises (i) at least one dose amount of any drug in the set of drugs and (ii) a recommended schedule for administering the at least one dose amount to the patient, the recommended schedule including a recommended time for administering a next dose to the patient, such that a predicted concentration time profile of any drug in the set of drugs in the patient in response to the first pharmaceutical dosing regimen is at or above the target drug exposure level at the recommended time;
(d) after a start of administration of a dosing regimen based at least in part on the first pharmaceutical dosing regimen to the patient, receiving additional concentration data and/or additional physiological data obtained from the patient;
(e) updating the inputs, based on the physiological data and the additional physiological data indicating a decline in health of the patient, to (i) exclude the concentration data from the inputs and (ii) include the additional physiological data in the inputs;
(f) updating, based on the updated inputs, the parameters for the computational model; and
(g) determining, using the computational model and the updated parameters, a second pharmaceutical dosing regimen for the patient.

2. (canceled)

3. The method of claim 1, wherein the updating the inputs occurs when the additional concentration data is consistent with the concentration data.

4. The method of claim 1, wherein the updated inputs:
(i) include the physiological data, the additional physiological data, and the target drug exposure level, and
(ii) exclude the concentration data and the additional concentration data.

5. (canceled)

6. (canceled)

7. (canceled)

8. (canceled)

9. (canceled)

10. (canceled)

11. (canceled)

12. The method of claim 1, further comprising receiving historical data indicative of a response of the patient to a previously administered drug of the set of drugs, wherein the computational model accounts for the historical data in order to generate predictions of concentration time profiles of the drug in the patient.

13. (canceled)

14. (canceled)

15. The method of claim 1, wherein the set of drugs is one of: monoclonal antibodies and antibody constructs, cytokines, drugs used for enzyme replacement therapy, aminoglycoside antibiotics, and chemotherapeutic agents that cause white cell decreases.

16. The method of claim 1, wherein each drug in the set of drugs is used to treat at least one of: an inflammatory disease, inflammatory bowel disease (IBD), rheumatoid arthritis, ankylosing spondylitis, psoriatic arthritis, psoriasis, asthma, or multiple sclerosis.

17. (canceled)

18. The method of claim 1, wherein the drug is infliximab or adalimumab.

19. (canceled)

20. The method of claim 1, wherein the inputs further include drug data comprising a route of administration, wherein the drug data excludes information identifying the specific drug belonging to the plurality of drugs, and wherein the route of administration is at least one of: subcutaneous, intravenous, oral, intramuscular, intrathecal, sublingual, buccal, rectal, vaginal, ocular, nasal, inhalation, nebulization, cutaneous, and transdermal.

21. The method of claim 20, wherein the drug data further comprises an available dosage unit for the specific drug belonging to the plurality of drugs, and wherein the dose amount is an integer multiple of the available dosage unit for certain routes of administration.

22. (canceled)

23. A method of determining a patient-specific pharmaceutical dosing regimen for a patient, using a computerized pharmaceutical dosing regimen recommendation system, the method comprising:
(a) receiving inputs including (i) concentration data indicative of one or more concentration levels of a drug in one or more samples obtained from the patient, wherein the drug is one of a set of drugs expected to exhibit similar pharmacokinetic (PK) behavior, similar pharmacodynamic (PD) behavior, or both, (ii) physiological data indicative of one or more measurements of at least one physiological parameter of the patient, and (iii) a target drug exposure level;
(b) determining, based on the received inputs, parameters for a computational model that generates predictions of concentration time profiles of the drug in the patient, wherein the computational model is representative of responses by a plurality of patients to a plurality of drugs in the set of drugs, wherein each response of the responses is indicative of a patient response to at least one drug in the set of drugs, and wherein the computational model is not specific to a particular drug;
(c) determining, using the computational model and based on the determined parameters, a first pharmaceutical dosing regimen for the patient, wherein the first pharmaceutical dosing regimen comprises (i) at least one dose amount of any drug in the set of drugs and (ii) a recommended schedule for administering the at least one dose amount any drug in the set of drugs to the patient, the recommended schedule including a recommended time for administering a next dose of any drug in the set of drugs to the patient, such that a predicted concentration time profile of any drug in the set of drugs in the patient in response to the first pharmaceutical dosing regimen is at or above the target drug exposure level at the recommended time;
(d) after a start of administration of a dosing regimen based at least in part on the first pharmaceutical dosing regimen to the patient, receiving additional concentration data and additional physiological data obtained from the patient;
(e) updating the concentration data to include the concentration data and the additional concentration data;
(f) dividing the updated concentration data into a subset and a remaining portion;
(g) updating the inputs, based on the updated concentration data indicating a material change in the concentration level of the drug in the patient, to (i) include the additional physiological data in the inputs, (ii) include the subset of the updated concentration data in the inputs, and (iii) exclude the remaining portion of the updated concentration data from the inputs;
(h) updating, based on the updated inputs, the parameters for the computational model; and
(i) determining, using the computational model and the updated parameters, a second pharmaceutical dosing regimen for the patient.

24. The method of claim 23, wherein the subset of the updated concentration data consists of up to three most recent data points in the updated concentration data.

25. The method of claim 23, wherein dividing the updated concentration data into a subset and a remaining portion is based on whether an administered period of treatment of the first pharmaceutical dosing regimen is greater than a proportion of a total length of time of the first pharmaceutical dosing regimen.

26. The method of claim 25, wherein the subset of the updated concentration data consists of one most recent data point in the updated concentration data and the additional concentration data.

27. The method of claim 23, wherein the subset is determined based on whether the physiological data and the additional physiological data indicate a decline in health of the patient, and wherein when the physiological data and the additional physiological data are indicative of the decline in health of the patient, the subset consists of up to three most recent data points in the updated concentration data.

28. (canceled)

29. The method of claim 23, wherein the subset is determined based on whether the physiological data and the additional physiological data indicate a decline in health of the patient, and further comprising determining, when the physiological data and the additional physiological data do not indicate the decline in health of the patient, whether the additional concentration data is an anomaly.

30. The method of claim 29, wherein:
when the additional concentration data is an anomaly, the remaining portion consists of the additional concentration data, and
when the additional concentration data is not an anomaly, the subset consists of up to three most recent data points in the updated concentration data.

31. The method of claim 23, wherein each drug in the set of drugs is used to treat at least one of: an inflammatory disease, inflammatory bowel disease (IBD), rheumatoid arthritis, ankylosing spondylitis, psoriatic arthritis, psoriasis, asthma, or multiple sclerosis.

32. (canceled)

33. The method of claim 23, wherein the drug is infliximab or adalimumab.

34. (canceled)

35. The method of claim 23, wherein the inputs further include drug data comprising a route of administration and an available dosage unit for the drug belonging to the plurality of drugs, the drug data excluding information identifying the drug belonging to the plurality of drugs, wherein the dose amount is an integer multiple of the available dosage unit for certain routes of administration, and wherein the route of administration is at least one of: subcutaneous, intravenous, oral, intramuscular, intrathecal, sublingual, buccal, rectal, vaginal, ocular, nasal, inhalation, nebulization, cutaneous, and transdermal.

36. (canceled)

37. (canceled)

38. A method of determining a patient-specific pharmaceutical dosing regimen for a patient, using a computerized pharmaceutical dosing regimen recommendation system, the method comprising:
(a) receiving inputs including (i) prior concentration data indicative of one or more prior concentration levels of a historical drug in one or more samples obtained from the patient, (ii) physiological data indicative of one or more measurements of at least one physiological parameter of the patient, and (iii) a target drug exposure level of a current drug, wherein the current drug is one of a set of drugs expected to exhibit similar pharmacokinetic (PK) behavior, similar pharmacodynamic (PD) behavior, or both;
(b) determining, based on the received inputs, parameters for a computational model that generates predictions of concentration time profiles of the current drug in the patient, wherein the computational model is representative of responses by a plurality of patients to a plurality of drugs in the set of drugs, wherein each response of the responses is indicative of a patient response to at least one drug in the set of drugs, and wherein the computational model is not specific to a particular drug;
(c) determining, using the computational model and based on the determined parameters, a first pharmaceutical dosing regimen for the patient, wherein the first pharmaceutical dosing regimen comprises (i) at least one dose amount of any drug in the set of drugs and (ii) a recommended schedule for administering the at least one dose amount of any drug in the set of drugs to the patient, the recommended schedule including a recommended time for administering a next dose of any drug in the set of drugs to the patient, such that a predicted concentration time profile of any drug in the set of drugs in the patient in response to the first pharmaceutical dosing regimen is at or above the target drug exposure level at the recommended time;
(d) after a start of administration of a dosing regimen based at least in part on the first pharmaceutical dosing regimen to the patient, receiving additional concentration data indicative of one or more concentration levels of the current drug in one or more samples obtained from the patient and additional physiological data obtained from the patient;
(g) updating the inputs to (i) include the additional physiological data in the inputs and (ii) include the additional concentration data in the inputs;
(h) updating, based on the updated inputs, the parameters for the computational model; and
(i) determining, using the computational model and the updated parameters, a second pharmaceutical dosing regimen for the patient.

39. (canceled)

40. (canceled)
